(19) United States
(12) Patent Application Publication
(10) Pub. No.: US 2024/0102099 A1
(43) Pub. Date: Mar. 28, 2024

(54) METHODS AND COMPOSITIONS RELATING TO A NOVEL EPIDERMAL GROWTH FACTOR RECEPTOR (EGFR) SPLICE VARIANT

(71) Applicant: H. LEE MOFFITT CANCER CENTER AND RESEARCH INSTITUTE, INC., Tampa, FL (US)

(72) Inventors: Brandon J. MANLEY, Tampa, FL (US); Timothy J. ROBINSON, Madison, CT (US); Anthony MAGLIOCCO, Richmond (CA); Liang WANG, Tampa, FL (US)

(21) Appl. No.: 18/037,915

(22) PCT Filed: Nov. 22, 2021
(86) PCT No.: PCT/US2021/060276

§ 371 Date: May 19, 2023

Related U.S. Application Data
(60) Provisional application No. 63/116,197, filed on Nov. 20, 2020.
(60) Provisional application No. 63/242,866, filed on Sep. 10, 2021.

(51) Int. Cl. C12Q 1/6886; A61P 35/04; C07K 16/28

(52) U.S. Cl. CPC C12Q 1/6886; A61P 35/04; C07K 16/2818; C07K 2317/21; C07K 2317/24; C12Q 2600/106; C12Q 2600/158

(57) ABSTRACT

Disclosed are methods for detecting resistance to immune checkpoint blockade inhibition in cancer patients expressing a epidermal growth factor receptor (EGFR) splice variant.

### I. BACKGROUND

Our current understanding of actionable genomic alterations in ccRCC, the most common type of kidney cancer, and their role in managing ccRCC remains sparse compared to that of other malignancies. In the majority of patient interactions, specific mutations in ccRCC and the disease's mutational burden have not proven to be clinically impactful. Despite this focus on mutational burden, diagnostic, prognostic, and therapeutic targets for kidney cancer are relatively nonexistent. EGFR_PR20CTF, a novel epidermal growth factor receptor aberrant splice variant that is unique to clear cell renal cell carcinoma (ccRCC) has been identified. EGFR_PR20CTF is present in approximately 50% to 60% of all ccRCC tumors. The high frequency of aberrant EGFR_PR20CTF splicing observed in ccRCC and its association with more advanced disease makes it an exciting discovery, given the broad applicability it has as a biomarker and a treatment target. Investigation into the biology of EGFR_PR20CTF can lead to the development of tissue, blood, or urine tests to yield gene or protein biomarkers of EGFR_PR20CTF in ccRCC.

### II. SUMMARY

Disclosed are methods and compositions for detecting resistance to immune checkpoint blockade inhibition in cancer patients with decreased expression of an epidermal growth factor receptor (EGFR) splice variant, Family With Sequence Similarity 107 Member B (FAM107B) splice variant, PDZ Domain Containing 2 (PDZD2) splice variant, Cordon-Bleu WH2 Repeat Protein Like 1 (COBLL1) splice variant, faciogenital dysplasia 1 (FGD1) splice variant, Galactose-3-O-Sulfotransferase 1 (GAL3ST1) splice variant, Hippocalcin Like 1 (HPCAL1) splice variant, KDEL (Lys-Asp-Glu-Leu) Containing 1 (KDELC1) splice variant, Methylcrotonyl-CoA Carboxylase Subunit 1 (MCCC1) splice variant, mevalonate kinase (MVK) splice variant, MVKb splice variant, Poly(A)-Specific Ribonuclease (PARN) splice variant, Protein Tyrosine Phosphatase Non-Receptor Type 14 (PTPN14) splice variant, Ribonuclease T2 beta (RNASET2β) splice variant, Solute carrier family 15, member 4 (SLC15A4) splice variant, and/or Synaptopodin (SYNPO) splice variant.

In one aspect, disclosed herein are methods of detecting resistance of a cancer (such as, for example, kidney (i.e., renal) cancer) to immune checkpoint blockade inhibition (such as for example, anti-PD-L1 inhibition such as MDX-1105 (BMS-936559), MPDL3280A, or MSB0010718C) comprising obtaining a tissue sample (such as, for example, whole blood, serum, plasma, cerebrospinal fluid, or urine) from a subject with a cancer, performing droplet digital polymerase chain reaction (ddPCR) on the tissue sample comprising amplifying nucleic acid (including, but not limited to cell free DNA and/or RNA) using primers specific for an epidermal growth factor receptor (EGFR) splice variant (such as, for example, a splice variant isoform that has a deletion of the EGF binding domain including, but not limited to an alpha splice isoform of EGFR, a beta splice isoform of EGFR, or a gamma splice isoform of EGFR), Family With Sequence Similarity 107 Member B (FAM107B) splice variant, PDZ Domain Containing 2 (PDZD2) splice variant, Cordon-Bleu WH2 Repeat Protein Like 1 (COBLL1) splice variant, faciogenital dysplasia 1 (FGD1) splice variant, Galactose-3-O-Sulfotransferase 1 (GAL3ST1) splice variant, Hippocalcin Like 1 (HPCAL1) splice variant, KDEL (Lys-Asp-Glu-Leu) Containing 1 (KDELC1) splice variant, Methylcrotonyl-CoA Carboxylase Subunit 1 (MCCC1) splice variant, mevalonate kinase (MVK) splice variant, MVKb splice variant, Poly(A)-Specific Ribonuclease (PARN) splice variant, Protein Tyrosine Phosphatase Non-Receptor Type 14 (PTPN14) splice variant, Ribonuclease T2 beta (RNASET2β) splice variant, Solute carrier family 15, member 4 (SLC15A4) splice variant, and/or Synaptopodin (SYNPO) splice variant; wherein the decrease in the expression of an EGFR, FAM107B, PDZD2, COBLL1, FGD1, GAL3ST1, HPCAL1, KDELC1, MCCC1, MVK, MVKβ, PARN, PTPN14, RNASET2β, SLC15A4, and/or SYNPO splice variant in the tissue sample indicates that the cancer is resistant to immune checkpoint blockade inhibition (such as for example, anti-PD-L1 inhibition such as MDX-1105 (BMS-936559), MPDL3280A, or MSB0010718C). In some aspect, the methods of detecting resistance of a cancer to immune checkpoint blockade inhibitors can further comprise treating the subject with an anti-cancer therapy that is not an immune checkpoint blockade inhibitor when an EGFR, FAM107B, PDZD2, COBLL1, FGD1, GAL3ST1, HPCAL1, KDELC1, MCCC1, MVK, MVKβ, PARN, PTPN14, RNASET2β, SLC15A4, and/or SYNPO splice variant is detected or administering an immune checkpoint blockade inhibitor when an EGFR splice variant is not detected. In some aspects, the methods of detecting resistance of a cancer to immune checkpoint blockade inhibitors can further comprise treating the subject with one or more immune checkpoint blockade inhibitors that do not target PD-L1 (such as for example, an immune checkpoint inhibitor that targets PD-1 (such as, for example, Pembrolizumab and/or Nivolumab), PD-L2, CRLA-4 (such as, for example, Ipilimumab), IDO, B7-H3, B7-H4, TIM3, or LAG-3) when an EGFR, FAM107B, PDZD2, COBLL1, FGD1, GAL3ST1, HPCAL1, KDELC1, MCCC1, MVK, MVKβ, PARN, PTPN14, RNASET2β, SLC15A4, and/or SYNPO splice variant is detected.

Also disclosed herein are methods of treating, inhibiting, reducing, decreasing, ameliorating, and/or preventing a cancer (such as, for example, kidney cancer) and/or metastasis in a subject with a cancer comprising obtaining a tissue sample (such as, for example, whole blood, serum, plasma, cerebrospinal fluid, or urine) from the subject, performing droplet digital polymerase chain reaction (ddPCR) on the tissue sample comprising amplifying nucleic acid (including, but not limited to cell free DNA and/or RNA) using primers specific for an epidermal growth factor receptor (EGFR) splice variant (such as, for example, a splice variant isoform that has a deletion of the EGF binding domain including, but not limited to an alpha splice isoform of EGFR, a beta splice isoform of EGFR, or a gamma splice isoform of EGFR), Family With Sequence Similarity 107 Member B (FAM107B) splice variant, PDZ Domain Containing 2 (PDZD2) splice variant, Cordon-Bleu WH2 Repeat Protein Like 1 (COBLL1) splice variant, faciogenital dysplasia 1 (FGD1) splice variant, Galactose-3-O-Sulfotransferase 1 (GAL3ST1) splice variant, Hippocalcin Like 1 (HPCAL1) splice variant, KDEL (Lys-Asp-Glu-Leu) Containing 1 (KDELC1) splice variant, Methylcrotonyl-CoA Carboxylase Subunit 1 (MCCC1) splice variant, mevalonate kinase (MVK) splice variant, MVKb splice variant, Poly(A)-Specific Ribonuclease (PARN) splice variant, Protein Tyrosine Phosphatase Non-Receptor Type 14 (PTPN14) splice variant, Ribonuclease T2 beta (RNASET2β) splice variant, Solute carrier family 15, member 4 (SLC15A4) splice variant, and/or Synaptopodin (SYNPO) splice variant; wherein a decrease in the expression of an EGFR, FAM107B, PDZD2, COBLL1, FGD1, GAL3ST1, HPCAL1, KDELC1, MCCC1, MVK, MVKβ, PARN, PTPN14, RNASET2β, SLC15A4, and/or SYNPO splice variant in the tissue sample indicates that the cancer is resistant to immune checkpoint blockade inhibition (such as for example, anti-PD-L1 inhibition such as MDX-1105 (BMS-936559), MPDL3280A, or MSB0010718C); and administering an anti-cancer therapy when EGFR, FAM107B, PDZD2, COBLL1, FGD1, GAL3ST1, HPCAL1, KDELC1, MCCC1, MVK, MVKβ, PARN, PTPN14, RNASET2β, SLC15A4, and/or SYNPO splice variant is detected. In some aspects, the anti-cancer agent used in any of the preceding methods of treating, inhibiting, reducing, decreasing, ameliorating, and/or preventing a cancer and/or metastasis does not comprise an immune checkpoint blockade inhibitor. In some aspect, the anti-cancer agent used in any of the preceding methods of treating, inhibiting, reducing, decreasing, ameliorating, and/or preventing a cancer and/or metastasis comprises administering the subject with one or more immune checkpoint blockade inhibitors that do not target PD-L1 (such as for example, an immune checkpoint inhibitor that targets PD-1 (such as, for example, Pembrolizumab and/or Nivolumab), PD-L2, CRLA-4 (such as, for example, Ipilimumab), IDO, B7-H3, B7-H4, TIM3, or LAG-3) when an EGFR, FAM107B, PDZD2, COBLL1, FGD1, GAL3ST1, HPCAL1, KDELC1, MCCC1, MVK, MVKβ, PARN, PTPN14, RNASET2β, SLC15A4, and/or SYNPO splice variant is detected.

In one aspect, disclosed herein are methods of detecting the presence of a metastatic cancer (such as, for example, metastatic renal cancer) in a subject comprising obtaining a tissue sample (such as, for example, whole blood, serum, plasma, cerebrospinal fluid, or urine) from the subject, performing droplet digital polymerase chain reaction (ddPCR) on the tissue sample comprising amplifying nucleic acid (including, but not limited to cell free DNA and/or RNA) using primers specific for an epidermal growth factor receptor (EGFR) splice variant, Family With Sequence Similarity 107 Member B (FAM107B) splice variant, PDZ Domain Containing 2 (PDZD2) splice variant, Cordon-Bleu WH2 Repeat Protein Like 1 (COBLL1) splice variant, faciogenital dysplasia 1 (FGD1) splice variant, Galactose-3-O-Sulfotransferase 1 (GAL3ST1) splice variant, Hippocalcin Like 1 (HPCAL1) splice variant, KDEL (Lys-Asp-Glu-Leu) Containing 1 (KDELC1) splice variant, Methylcrotonyl-CoA Carboxylase Subunit 1 (MCCC1) splice variant, mevalonate kinase (MVK) splice variant, MVKb splice variant, Poly(A)-Specific Ribonuclease (PARN) splice variant, Protein Tyrosine Phosphatase Non-Receptor Type 14 (PTPN14) splice variant, Ribonuclease T2 beta (RNASET2β) splice variant, Solute carrier family 15, member 4 (SLC15A4) splice variant, and/or Synaptopodin (SYNPO) splice variant; wherein a decrease in the expression of an EGFR, FAM107B, and/or PDZD2 splice variant in the tissue sample indicates that the cancer has metastasized. In some aspect, the methods of detecting a metastatic cancer can further comprise treating the subject with an anti-cancer therapy that is not an immune checkpoint blockade inhibitor when an EGFR, FAM107B, PDZD2, COBLL1, FGD1, GAL3ST1, HPCAL1, KDELC1, MCCC1, MVK, MVKβ, PARN, PTPN14, RNASET2β, SLC15A4, and/or SYNPO splice variant is detected or administering an immune checkpoint blockade inhibitor when an EGFR splice variant is not detected. In some aspects, the methods of d detecting a metastatic cancer can further comprise treating the subject with one or more immune checkpoint blockade inhibitors that do not target PD-L1 (such as for example, an immune checkpoint inhibitor that targets PD-1 (such as, for example, Pembrolizumab and/or Nivolumab), PD-L2, CRLA-4 (such as, for example, Ipilimumab), IDO, B7-H3, B7-H4, TIM3, or LAG-3) when an EGFR, FAM107B, PDZD2, COBLL1, FGD1, GAL3ST1, HPCAL1, KDELC1, MCCC1, MVK, MVKβ, PARN, PTPN14, RNASET2β, SLC15A4, and/or SYNPO splice variant is detected.

Also, disclosed herein are methods of detecting recurrence of a cancer (such as, for example, renal cancer) following immunotherapy, chemotherapy, radiation, and or tissue resection, the method comprising obtaining a tissue sample (such as, for example, whole blood, serum, plasma, cerebrospinal fluid, or urine) from the subject, performing droplet digital polymerase chain reaction (ddPCR) on the tissue sample comprising amplifying nucleic acid (including, but not limited to cell free DNA and/or RNA) using primers specific for an epidermal growth factor receptor (EGFR) splice variant, Family With Sequence Similarity 107 Member B (FAM107B) splice variant, PDZ Domain Containing 2 (PDZD2) splice variant, Cordon-Bleu WH2 Repeat Protein Like 1 (COBLL1) splice variant, faciogenital dysplasia 1 (FGD1) splice variant, Galactose-3-O-Sulfotransferase 1 (GAL3ST1) splice variant, Hippocalcin Like 1 (HPCAL1) splice variant, KDEL (Lys-Asp-Glu-Leu) Containing 1 (KDELC1) splice variant, Methylcrotonyl-CoA Carboxylase Subunit 1 (MCCC1) splice variant, mevalonate kinase (MVK) splice variant, MVKb splice variant, Poly(A)-Specific Ribonuclease (PARN) splice variant, Protein Tyrosine Phosphatase Non-Receptor Type 14 (PTPN14) splice variant, Ribonuclease T2 beta (RNASET2β) splice variant, Solute carrier family 15, member 4 (SLC15A4) splice variant, and/or Synaptopodin (SYNPO) splice variant; wherein a decrease in the expression of an EGFR, FAM107B, PDZD2, COBLL1, FGD1, GAL3ST1, HPCAL1, KDELC1, MCCC1, MVK, MVKβ, PARN, PTPN14, RNASET2β, SLC15A4, and/or SYNPO splice variant in the tissue sample indicates that the cancer has recurred. In some aspects, the method can further comprise adjusting the treatment regimen, changing a treatment regimen, or recommencing a treatment regimen upon detection of cancer recurrence.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows an exon map for EGFR splice variant C-terminus fragment isoforms. No 50 splicing events are observed in these alternate forms, suggesting that they may be the beginning of a novel transcript. The second codon in exon 21, codon 825, is a start codon that encodes the amino acid methionine (g.7:55259415-55259417), indicating that a protein product could arise from these novel splice forms, starting with M825 (EGFR NM_005228/NP_005219 1210 amino acids). This protein product would include the 386 C-terminal amino acids, including part of the kinase domain (region PTKc_EGFR, CDD:270683, 704-1016, M825 is at codon position 122 of 313 codons in this domain). EGFR=epidermal growth factor receptor; mRNA=messenger RNA; TKF=tyrosine kinase fragment.

FIG. 2 shows the distribution of the EGFR splice variant C-terminus fragment isoforms across Moffitt Total Cancer Care cohort renal cell carcinoma samples. A fraction of each EGFR 50 exon 21 splice junction that is represented by the EGFR splice variant C-terminus fragment isoforms, alpha (black), beta (dark gray), and gamma (light gray), is plotted for each patient in the Moffitt Total Cancer Care cohort (n=99). EGFR=epidermal growth factor receptor.

FIGS. 3A, 3B, 3C, 3D, 3E, and 3F show the distribution of the EGFR splice variant C-terminus fragment isoforms across The Cancer Genome Atlas (TCGA) kidney samples. A fraction of each EGFR 50 Exon 21 splice junction that is represented by the EGFR splice variant C-terminus fragment isoforms, alpha (black), beta (dark gray), and gamma (light gray), are plotted for each patient in the TCGA cohort: FIG. 3A shows TCGA kidney renal clear cell carcinoma tumor samples (n=492), FIG. 3B shows TCGA kidney renal papillary cell carcinoma tumor samples (n=190), (3C) TCGA KICH tumor samples (n=50), (3D) TCGA kidney renal clear cell carcinoma adjacent normal samples (n=69), (3E) TCGA kidney renal papillary cell carcinoma adjacent normal samples (n=28), and (3F) TCGA KICH adjacent normal samples (n=25). EGFR=epidermal growth factor receptor; KICH=chromophobe kidney cancer; KIRC=kidney renal clear cell carcinoma; KIRP=kidney renal papillary cell carcinoma.

FIGS. 4A, 4B, and 4C show the validation of EGFR_pr20CTF expression. Gene expression results of cDNA isolated from patient tissue samples and cell lines using primers amplifying the novel EGFR-20-CTFa splice isoform junction: FIG. 4A shows RT-PCR results from patient samples; FIG. 4B shows samples used, fraction EGFR_pr20CTFa splice isoform junction as determined by RNAseq, and tissue types represented in FIG. 4A; and (4C) RT-PCR results from indicated cell lines. EGFR=epidermal growth factor receptor; EGFR_pr20CTF=EGFR splice variant C-terminus fragment starting from novel exon 20; RCC=renal cell carcinoma; RT-PCR=reverse transcriptase-polymerase chain reaction.

FIG. 5 shows a Kaplan-Meier curves for recurrence free survival analysis in ccRCC patients (n=62) using 1% expression cutoff values (High vs. Low) for presence of EGFR-20-CTF.

FIGS. 6A and 6B show the response to immunotherapy and targeted therapy stratified by EGFR level. KM curves of treatment response by tertiles of EGFR_pr20CTF expression. FIG. 6A shows that patients who received targeted therapy (n=29) did not demonstrate a statistical difference in the TCC ccRCC cohort. FIG. 6B shows that patients who received immunotherapy (n=19) demonstrated a statistical difference in overall survival from time on first immunotherapy (p=0.03). ccRCC=clear cell renal cell carcinoma; EGFR=epidermal growth factor receptor; TCC=Total Cancer Care Protocol.

FIG. 7 shows a duplex RT-PCR assay to quantify EGFR wildtype and splicing variant.

FIGS. 8A and 8B shows Kaplan-Meier survival curves for overall survival from the cancer genome atlas clear cell renal cell carcinoma (TCGA-KIRC) for splice variant PDZD2 (8A) and FAM107B (8B). (blue=double negative, red=dual positive)

FIG. 8C shows stratification of patients into quartiles based on RNASET2 splice variants presence (blue=double negative, red=dual positive).

FIG. 9A shows digitally derived multiplexed immunofluorescence ccRcc images from tumor stroma interface high tumor/CD163+ clustering indicating superior survival.

FIG. 9B shows similar image from ccRCcc tumor with low tumor/CD163+ clustering indicating inferior survival.

FIG. 9C shows Kaplan-Meir survival curve from the Moffit ccRcc patients (n=97) stratified for tumor/CD163+ clustering at radii of 75 microns (nK(75)) using a median cut point by high and low tumor/CD163+ clustering.

FIG. 9D shows a measurement of tumor/CD163+ clustering at tumor-stromal interface by RECIST 1.1 best overall response for 9 patient treatment with immunotherapy for metastatic ccRcc. Positive values on x-axis indicate above expected clustering of tumor cells with CD163+ cells. Negative values indicate above expected dispersion of tumor cells from CD163+.

FIGS. 10A and 10B show FAM107B splice variant detection (copies per nanogram=co/ng) in RNA from tissue and plasma from a patient with clear cell renal cell carcinoma. Prostate adenocarcinoma cell line (PC3) and kidney carcinoma cell line (A498) were used as negative and positive controls, respectively. Tissue cDNA (10A) was used to establish FAM107B splice variant detection in tumor tissue. Matched pre-surgery patient plasma sample was divided into 2 wells (10B) to increase sensitivity of the QIAcuity dPCR 26K plaste.

FIG. 11 shows a schematic of the screening and selection of splice variants and the 16 splice variants identified.

FIG. 12 shows the correlation between splice variants and wild type gene expression in cancerous tissue.

FIGS. 13A and 13B shows the hazard ratio (13A) and a clustering heatmap (13B) for splice variants.

FIG. 14 shows cancer specific survival associated with the expression versus non-expression of certain splice variants.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular EGFR splice variant is disclosed and discussed and a number of modifications that can be made to a number of molecules including the EGFR splice variant are discussed, specifically contemplated is each and every combination and permutation of EGFR splice variant and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Since the completion of the Human Genome Project, there has been increasing interest in the large discrepancy between the number of human genes (˜20 000) and the total number of distinct proteins expressed in humans (>150 000). Alternative mRNA splicing is one of the main processes that allows for this difference to occur due to its rearrangement and selective utilization of existing coding and noncoding sequences. Alternative mRNA splicing can also become altered or even pathologic in specific disease settings, including cancer; aberrant mRNA splicing can result in completely new, otherwise unexpressed mRNA isoforms, as in the expression of novel isoforms with altered functions. The most well-known aberrant mRNA splicing event is the ARv7 aberrant mRNA isoform. This isoform, which is expressed in castration-resistant metastatic prostate cancer, activates androgen receptor signaling in the absence of ligands and strongly predicts androgen-therapy resistance. Other examples of aberrant mRNA splicing events include MET exon 14 splicing mutations in lung cancer; mutations activating a cryptic splice site that result in an active form of NOTCH1 in chronic lymphocytic leukemia and EGFRvIII in glioblastomas.

Current understandings of actionable genomic mutations in kidney cancer and their role in managing this disease remain sparse compared to that of other malignancies. This appears to be due to the low overall mutational burden of these tumors, which have been the primary focus of modern genomic assays and the basis for understanding and managing genomic mutations in other cancers. As a result of this focus on mutational burden, diagnostic, prognostic, and therapeutic targets for kidney cancer are relatively nonexistent, and the study of potential biomarkers, drivers, and therapeutic targets of kidney cancer, with respect to alternative and aberrant mRNA splicing, is lacking.

Clinicians currently lack a blood-based biomarker(s) for ccRCC that could be applied across the spectrum of disease management in a similar role as prostate-specific antigen (PSA) for prostate cancer, for which PSA acts as a critical serum biomarker but not a specific drug-targeted protein. Other applications include the possible use of the splice variants as a prognostic or diagnostic panel, similar to those employed using gene expression profiling (i.e. Oncotype, Polaris) in other tumor types. Limitations of prior efforts to identify actionable liquid biopsy biomarkers for ccRCC include the overall low mutational burden, heterogeneity of molecular alterations (no hotspot mutations), and dependence on the epithelial cell adhesion molecule, which is poorly expressed in ccRCC.

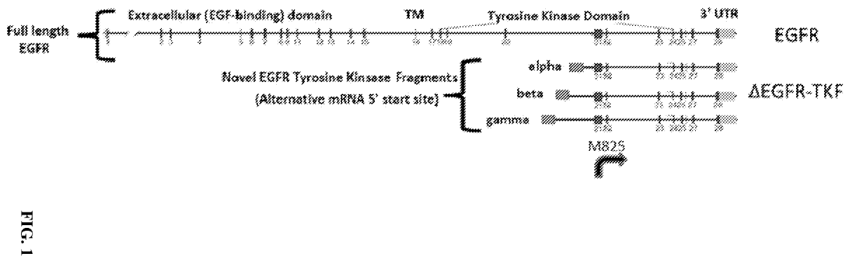

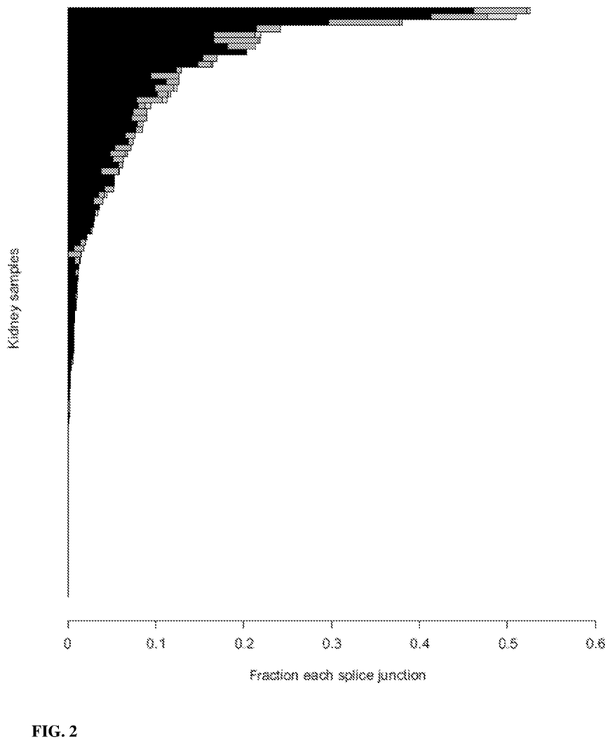

In this study, a recently identified novel epidermal growth factor receptor (EGFR) aberrant splicing variant is described that is unique to clear cell renal cell carcinoma (ccRCC) and present in up to 50% to 60% of all patients in the multi-institutional cohort of patients with a range of local and metastatic disease. The aberrant EGFR_PR20CTF in ccRCC is shown in FIG. 1. Three different splice forms were observed, which were labeled as alpha, beta, and gamma on the basis of their increasing distances upstream from the exon 21 50 binding site. The α-isoform is the predominate isoform, occurring in >95% all tumors that demonstrate EGFR_PR20CTF. The distribution of the EGFR_pr20CTF iso-forms across is detailed in FIG. 2.

The data shown herein has revealed several exciting characteristics of EGFR_PR20CTF in relation to its frequency, specificity and associations with important clinical outcomes like tumor recurrence and response to immunotherapy treatment. Gene Set Enrichment Analysis (GESA) using HALLMARK and Oncogenic gene sets (C6) has demonstrated increased expression of several key signaling genes among tumors with EGFR_PR20CTF including: KRAS (FDR q-val=0.035); AKT (FDR q-val=0.022) and HALLMARK KRAS (FDR q-val=0.023). This data helps support the hypothesis that EGFR_PR20CTF is increasing signaling of several downstream pathways including EGFR/MAPK and MTORC. Additionally, the GESA analysis showed significant decrease expression for several gene sets related to immune response including: HALLMARK inflammatory response, IL-2 signaling and interferon gamma response (all FDR q-val=<0.001). These results point to a global decrease in immune response in tumors with EGFR_PR20CTF and is a reason for these patients decreased response to immunotherapy.

In order to examine the biological function and clinical impact of EGFR_PR20CTF, a combination of genomics and proteomics approaches can be leveraged. Experience investigating protein kinases in a number of cancer types provides a toolbox for examining the protein product of this EGFR splice variant and its role in ccRCC. In particular, the approach herein is informed by research into the biology and drug targeting of full-length EGFR in both wild-type (WT) and mutant forms. Previous projects include EGFR phosphorylation mapping, investigation of drug mechanisms of action, and phosphoproteomics of EGFR-targeted kinase inhibitor resistance. In addition, analyses of tyrosine kinase signaling in kidney cancers was completed, which provide additional background information for this project.

There are a variety of transcriptomic technologies that can be used in identifying and investigating splice variants. EGFR_PR20CTF is ideally suited for Droplet Digital PCR (ddPCR) given the known splice junctions of the alteration through the use of specific primers used in the RT-PCR experiments. Using ddPCR offers significant cost saving compared to RNA-seq, less labor and informatics expertise and quicker adoption to Clinical Laboratory Improvement Amendments (CLIA) regulations. Similar clinical applications are currently used to identify BRAF V600E patients.

The long-term objective of the proposal seeks to elucidate the biological and therapeutic implications of EGFR_PR20CTF in kidney cancer. Objectives specific to this proposal include characterizing the novel EGFR_PR20CTF protein product and its mechanistic and biological impact in ccRCC tumors. Additionally, EGFR_PR20CTF expression can be defined using ddPCR clinical and therapeutic associations of these variants in patients, specifically; primary resistance to first-line to immune checkpoint blockade (ICB) treatment. The results of the study provide information critical for implementing data-driven, biology-based clinical management decisions and treatment strategies in ccRCC.

EGFR_PR20CTF is expressed at the protein level, interacts with full length EGFR to promote carcinogenesis in ccRCC and is significantly correlated with increased risk of tumor recurrence and primary resistance to ICB treatment. The use of ddPCR is a practical testing method to identify such patients.

Validate presence and association of EGFR_PR20CTF expression in ccRCC tumors with increased risk of primary resistance to ICB regimens for first line treatment of metastatic disease. One objective is to investigate primary resistance to ICB regimens, defined by progression-free survival ([PFS]<15 weeks) requiring change in treatment. Tumor samples from patients with metastatic ccRCC who receive first line treatment with ICB can be used. Droplet Digital PCR can be used to characterize EGFR_PR20CTF expression in tumor samples. Secondary Objective(s) include analyze objective radiographic treatment response according to modified Response Evaluation Criteria in Solid Tumors, version 1.1 (iRECIST 1.1) guidelines and identify associations of this response with EGFR_PR20CTF expression; determine prevalence of EGFR_PR20CTF expression in metastatic tumors using core biopsy samples and ddPCR; identify associations of EGFR_PR20CTF expression levels with secondary resistance as defined by PFS≤30 weeks necessitating a change in treatment regimen; and identify associations between dose-limiting drug toxicities and the presence of EGFR_PR20CTF alterations.

Define novel protein products from EGFR_PR20CTF and examine signaling of EGFR_PR20CTF to Develop Drug Repurposing Strategies for ccRCC. Here is focused on the verification of protein expression for EGFR_PR20CTF and its consequences. The primary objective here is to detect the proteoform associated with EGFR_PR20CTF in cell lines and tumors and determine whether the presence of this protein variant affects kinase signaling cascades in ccRCC, which indicate that EGFR_PR20CTF can be developed as a therapeutic target. Secondary Objective(s) include determining the extent to which EGFR_PR20CTF interacts with full-length WT EGFR and the consequences this interaction might have on kinase signaling; performing phenotypic assays (eg, Matrigel invasion, colony formation) to see determine which processes are enhanced by EGFR_PR20CTF expression; and determining whether EGFR_PR20CTF expression can rescue ccRCC cells after siRNA knockdown or CRISPR knockout of full-length WT EGFR.

Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms.

Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

### C. METHODS OF DETECTION AND/OR TREATMENT OF A CANCER

In one aspect, disclosed herein are methods of detecting resistance of a cancer (such as, for example, kidney cancer) to immune checkpoint blockade inhibition (such as for example, anti-PD-L1 inhibition such as MDX-1105 (BMS-936559), MPDL3280A, or MSB0010718C) comprising obtaining a tissue sample (such as, for example, a fluid tissue sample including, but not limited to whole blood, serum, plasma, cerebrospinal fluid, or urine) from a subject with a cancer, performing droplet digital polymerase chain reaction (ddPCR) on the tissue sample comprising amplifying nucleic acid using primers specific for an epidermal growth factor receptor (EGFR) splice variant (such as, for example, a splice variant isoform that has a deletion of the EGF binding domain including, but not limited to an alpha splice isoform of EGFR, a beta splice isoform of EGFR, or a gamma splice isoform of EGFR), Family With Sequence Similarity 107 Member B (FAM107B) splice variant, and/or PDZ Domain Containing 2 (PDZD2) splice variant; wherein a decrease in the expression EGFR, FAM107B, and/or PDZD2 splice variant in the tissue sample indicates that the cancer is resistant to immune checkpoint blockade inhibition (such as for example, anti-PD-L1 inhibition such as MDX-1105 (BMS-936559), MPDL3280A, or MSB0010718C). In some aspect, the methods of detecting resistance of a cancer to immune checkpoint blockade inhibitors can further comprise treating the subject with an anti-cancer therapy that is not an immune checkpoint blockade inhibitor when an EGFR, FAM107B, and/or PDZD2 splice variant is detected or administering an immune checkpoint blockade inhibitor when an EGFR splice variant is not detected. In some aspects, the methods of detecting resistance of a cancer to immune checkpoint blockade inhibitors can further comprise treating the subject with one or more immune checkpoint blockade inhibitors that do not target PD-L1 (such as for example, an immune checkpoint inhibitor that targets PD-1 (such as, for example, Pembrolizumab and/or Nivolumab), PD-L2, CRLA-4 (such as, for example, Ipilimumab), IDO, B7-H3, B7-H4, TIM3, or LAG-3) when an EGFR, FAM107B, and/or PDZD2 splice variant is detected.

Also disclosed herein are methods of treating, inhibiting, reducing, decreasing, ameliorating, and/or preventing a cancer (such as, for example, kidney cancer) and/or metastasis in a subject with a cancer comprising obtaining a tissue sample (such as, for example, whole blood, serum, plasma, cerebrospinal fluid, or urine) from the subject, performing droplet digital polymerase chain reaction (ddPCR) on the tissue sample comprising amplifying nucleic acid using primers specific for an epidermal growth factor receptor (EGFR) splice variant (such as, for example, a splice variant isoform that has a deletion of the EGF binding domain including, but not limited to an alpha splice isoform of EGFR, a beta splice isoform of EGFR, or a gamma splice isoform of EGFR), Family With Sequence Similarity 107 Member B (FAM107B) splice variant, and/or PDZ Domain Containing 2 (PDZD2) splice variant; wherein a decrease in the expression of an EGFR, FAM107B, and/or PDZD2 splice variant in the tissue sample indicates that the cancer is resistant to immune checkpoint blockade inhibition (such as for example, anti-PD-L1 inhibition such as MDX-1105 (BMS-936559), MPDL3280A, or MSB0010718C); and administering an anti-cancer therapy when EGFR, FAM107B, and/or PDZD2 splice variant is detected. In some aspects, the anti-cancer agent used in any of the preceding methods of treating, inhibiting, reducing, decreasing, ameliorating, and/or preventing a cancer and/or metastasis does not comprise an immune checkpoint blockade inhibitor. In some aspect, the anti-cancer agent used in any of the preceding methods of treating, inhibiting, reducing, decreasing, ameliorating, and/or preventing a cancer and/or metastasis comprises administering the subject with one or more immune checkpoint blockade inhibitors that do not target PD-L1 (such as for example, an immune checkpoint inhibitor that targets PD-1 (such as, for example, Pembrolizumab and/or Nivolumab), PD-L2, CRLA-4 (such as, for example, Ipilimumab), IDO, B7-H3, B7-H4, TIM3, or LAG-3) when an EGFR, FAM107B, and/or PDZD2 splice variant is detected.

Typically, diagnosis of metastatic renal cancer in a patient occurs at a time point too late to provide a therapeutic benefit and treatment of the patient becomes limited to palliative care designed to comfort the patient until death inevitably occurs. However, it is understood and herein contemplated that through the use of the disclosed methods, metastatic cancer can be detected much earlier thereby providing the opportunity for a therapeutic benefit to be achieved from treatment. Thus, detection of metastatic cancer or cancer recurrence following the commencement or termination of a treatment is highly beneficial. In one aspect, disclosed herein are methods of detecting the presence of a metastatic cancer (such as, for example, metastatic renal cancer) in a subject comprising obtaining a tissue sample (such as, for example, whole blood, serum, plasma, cerebrospinal fluid, or urine) from the subject, performing droplet digital polymerase chain reaction (ddPCR) on the tissue sample comprising amplifying nucleic acid using primers specific for an epidermal growth factor receptor (EGFR) splice variant, Family With Sequence Similarity 107 Member B (FAM107B) splice variant, and/or PDZ Domain Containing 2 (PDZD2) splice variant; wherein a decrease in the expression of an EGFR, FAM107B, and/or PDZD2 splice variant in the tissue sample indicates that the cancer has metastasized. In some aspect, the methods of detecting a metastatic cancer can further comprise treating the subject with an anti-cancer therapy that is not an immune checkpoint blockade inhibitor when an EGFR, FAM107B, and/or PDZD2 splice variant is detected or administering an immune checkpoint blockade inhibitor when an EGFR splice variant is not detected. In some aspects, the methods of d detecting a metastatic cancer can further comprise treating the subject with one or more immune checkpoint blockade inhibitors that do not target PD-L1 (such as for example, an immune checkpoint inhibitor that targets PD-1 (such as, for example, Pembrolizumab and/or Nivolumab), PD-L2, CRLA-4 (such as, for example, Ipilimumab), IDO, B7-H3, B7-H4, TIM3, or LAG-3) when an EGFR, FAM107B, and/or PDZD2 splice variant is detected.

Also, disclosed herein are methods of detecting recurrence of a cancer (such as, for example, renal cancer) following immunotherapy, chemotherapy, radiation, and or tissue resection, the method comprising obtaining a tissue sample (such as, for example, whole blood, serum, plasma, cerebrospinal fluid, or urine) from the subject, performing droplet digital polymerase chain reaction (ddPCR) on the tissue sample comprising amplifying nucleic acid using primers specific for an epidermal growth factor receptor (EGFR) splice variant, Family With Sequence Similarity 107 Member B (FAM107B) splice variant, and/or PDZ Domain Containing 2 (PDZD2) splice variant; wherein a decrease in the expression of an EGFR, FAM107B, and/or PDZD2 splice variant in the tissue sample indicates that the cancer has recurred. In some aspects, the method can further comprise adjusting the treatment regimen, changing a treatment regimen, or recommencing a treatment regimen upon detection of cancer recurrence.

1. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice.

Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

2. Method of Treating Cancer

As noted above, the disclosed detection methods can be used to direct the appropriate treatment regimen for any disease where uncontrolled cellular proliferation occurs such as cancers. Thus, in one aspect, disclosed herein are methods of treating, inhibiting, reducing, decreasing, ameliorating, and/or preventing a cancer (such as, for example, kidney cancer) and/or metastasis in a subject with a cancer comprising obtaining a tissue sample (such as, for example, whole blood, serum, plasma, cerebrospinal fluid, or urine) from the subject, performing droplet digital polymerase chain reaction (ddPCR) on the tissue sample comprising amplifying nucleic acid using primers specific for an epidermal growth factor receptor (EGFR) splice variant (such as, for example, a splice variant isoform that has a deletion of the EGF binding domain including, but not limited to an alpha splice isoform of EGFR, a beta splice isoform of EGFR, or a gamma splice isoform of EGFR), Family With Sequence Similarity 107 Member B (FAM107B) splice variant, PDZ Domain Containing 2 (PDZD2) splice variant, Cordon-Bleu WH2 Repeat Protein Like 1 (COBLL1) splice variant, faciogenital dysplasia 1 (FGD1) splice variant, Galactose-3-O-Sulfotransferase 1 (GAL3ST1) splice variant, Hippocalcin Like 1 (HPCAL1) splice variant, KDEL (Lys-Asp-Glu-Leu) Containing 1 (KDELC1) splice variant, Methylcrotonyl-CoA Carboxylase Subunit 1 (MCCC1) splice variant, mevalonate kinase (MVK) splice variant, MVKb splice variant, Poly(A)-Specific Ribonuclease (PARN) splice variant, Protein Tyrosine Phosphatase Non-Receptor Type 14 (PTPN14) splice variant, Ribonuclease T2 beta (RNASET2β) splice variant, Solute carrier family 15, member 4 (SLC15A4) splice variant, and/or Synaptopodin (SYNPO) splice variant; wherein a decrease in the expression of an EGFR, FAM107B, PDZD2, COBLL1, FGD1, GAL3ST1, HPCAL1, KDELC1, MCCC1, MVK, MVKβ, PARN, PTPN14, RNASET2β, SLC15A4, and/or SYNPO splice variant in the tissue sample indicates that the cancer is resistant to immune checkpoint blockade inhibition (such as for example, anti-PD-L1 inhibition such as MDX-1105 (BMS-936559), MPDL3280A, or MSB0010718C); and administering an anti-cancer therapy when an EGFR, FAM107B, PDZD2, COBLL1, FGD1, GAL3ST1, HPCAL1, KDELC1, MCCC1, MVK, MVKβ, PARN, PTPN14, RNASET2β, SLC15A4, and/or SYNPO splice variant is detected. In some aspects, the anti-cancer agent used in any of the preceding methods of treating, inhibiting, reducing, decreasing, ameliorating, and/or preventing a cancer and/or metastasis does not comprise an immune checkpoint blockade inhibitor. In some aspect, the anti-cancer agent used in any of the preceding methods of treating, inhibiting, reducing, decreasing, ameliorating, and/or preventing a cancer and/or metastasis comprises administering the subject with one or more immune checkpoint blockade inhibitors that do not target PD-L1 (such as for example, an immune checkpoint inhibitor that targets PD-1 (such as, for example, Pembrolizumab and/or Nivolumab), PD-L2, CRLA-4 (such as, for example, Ipilimumab), IDO, B7-H3, B7-H4, TIM3, or LAG-3) when an EGFR, FAM107B, PDZD2, COBLL1, FGD1, GAL3ST1, HPCAL1, KDELC1, MCCC1, MVK, MVKβ, PARN, PTPN14, RNASET2β, SLC15A4, and/or SYNPO splice variant is detected.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

The disclosed treatments can include any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine 1131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine 1131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine 1131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Where an EGFR splice variant isoform is not detected, the treatment methods can include or further include checkpoint inhibitors include, but are not limited to antibodies that block PD-1 (Pembrolizumab, Nivolumab (BMS-936558 or MDX1106), CT-011, MK-3475), PD-L1 (MDX-1105 (BMS-936559), MPDL3280A, or MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016). Where the presence of an EGFR, FAM107B, and/pr PDZD2 splice variant isoform is decreased the treatment regimen implemented does not include a immune checkpoint blockade inhibitor. It is understood and herein recognized that a decrease of an EGFR, FAM107B, and/or PDZD2 splice variant isoform does not necessarily indicate that the cancer is resistant to all immune checkpoint blockade inhibitors. In one aspect, the detection of the EGFR splice variant isoform indicates resistance to PD-1, PD-L1, PD-12, CRLA-4, IDO, B7-H3, B7-H4, TIM3, or LAG-3. In one aspect, the detection of a decrease of the EGFR, FAM107B, PDZD2, COBLL1, FGD1, GAL3ST1, HPCAL1, KDELC1, MCCC1, MVK, MVKβ, PARN, PTPN14, RNASET2β, SLC15A4, and/or SYNPO splice variant isoform indicates resistance to PD-L1. Thus, when resistance is only to a particular form of immune checkpoint blockade inhibition (such as, for example PD-L1), other immune checkpoint blockade inhibitors (such as, for example, Pembrolizumab and/or Nivolumab), PD-L2, CRLA-4 (such as, for example, Ipilimumab), IDO, B7-H3, B7-H4, TIM3, or LAG-3) can still be used.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Determine Association of EGFR Splice Variants in ccRCC Tumors and Primary Resistance to ICB Regimens for First Line Treatment of Metastatic Disease

Physicians at some cancer centers perform an average of >300 surgical procedures annually in the treatment of patients with this disease. Unfortunately, a significant percentage of these patients develop metastatic disease because of their having high-risk features. This patient group adds to the approximately 30% of all patients with kidney cancer who present with metastatic disease. In line with the current standard of care, the majority of patients with metastatic disease are treated with immunotherapy-based regimens for first-line treatment, including nivolumab, pembrolizumab, and avelumab. All patients who are treated at MCC are presented with the opportunity to consent to the TCC protocol, which aims to study the molecular and histologic basis of all tumor types; see letter of support and documents. Under this protocol, patients with metastatic disease consent to tissue donation and the sequencing of their donated tumor tissue and blood (whole-exome tumor, tumor RNA, and germline).

a) Eligibility Criteria

Inclusion criteria include patients who have a diagnosis of metastatic ccRCC and have or plan to initiate first line systemic treatment with a qualifying immunotherapy based regimen. A qualifying ICB drug includes nivolumab, pembrolizumab or avelumab. Combinations of immunotherapy with other FDA-approved agents are allowed (i.e. tyrosine kinase inhibitors). Study participants can be identified in both a retrospective and prospective fashion. All participants have the same study inclusion and exclusion requirements. Potential prospective participants can be identified by the primary physician or research coordinator in the Genitourinary (GU) Oncology department and invited for eligibility screening and consenting to the TCC protocol. Dr. Jad Chahoud, an expert in systematic treatment for ccRCC patient and co-investigator on the study, assist in screening and identifying eligible patients, see letter of support. Study patients need to have a standard of care CT or MRI restaging study at 12 weeks (+/−3 weeks) to assess response and a clinic appointment to evaluate for toxicities. Further follow up and imaging can be continued to be conducted at standard of care intervals and at the discretion of the treating physician. Patients need to have sufficient tissue for RNA extraction from either biopsy or surgical tissue before starting treatment. Patients need to consent to the TCC protocol. Central review (MCC pathology) can be required to confirm diagnosis and histology. Patients are excluded if they have had an active history of any known second non-GU malignancy in the past 5 years and have not received curative treatment. Both primary tumor samples and tumor samples from metastatic sites are included. However, based on the historical frequency of sample acquisition at MCC, the majority of these samples with come from biopsy of metastatic lesions. Also, the prevalence and expression of EGFR_PR20CTF can be determined in core biopsy samples from metastatic lesions using a practical and clinically applicable ddPCR protocol. This objective is to provide the foundation for this testing in the clinical setting.

The analysis has been powered based off a prospective cohort alone but the inclusion of retrospective patients (n=25) allow for tissue based studies to commence upon funding. Their inclusion provide valuable controls for comparing transcriptomic technologies ddPCR vs. RNA-seq and EGFR_PR20CTF expression. These patients can also be used in the primary analysis. Considering a dropout rate of 10%, at least 77 subjects can be accrued in this prospective study. After confirming eligibility and consent patients can be enrolled in the study. For this study, there is no randomization and patients can be treated with the current standard of care at the discretion of their treating physician. There can be no blinding of patient or treatment data.

b) EGFR_PR20CTF Expression:

For prospectively collected tissues, fresh or frozen tumor samples can be collected and labeled through the institutional biobank under the TCC protocol. If frozen tumor samples are unavailable, then representative formalin-fixed, paraffin-embedded blocks can be used. Samples undergo RNA extraction and quality control can be performed per MCC Tissue Core standard protocols. Only samples that pass quality control-conducted using Nanodrop and/or Qubit quantification and assessment per standard tissue core extraction protocols go on for ddPCR. Extracted RNA samples can then undergo cDNA generation via reverse transcription. John Puskas (see letter) oversee this work. He currently runs a CLIA certified lab that uses these technologies to identify BRAF and EGFR variants, see letter. Specific primers for EGFR_PR20CTF most common variant, alpha, can be used; similar to those in the RT-PCR experiments. Primers and template can be sourced from the EvaGreen ddPCR Demonstration Kit (Bio-Rad: 186-4029). Primers can be diluted according to the kit instructions and template can be diluted by approximately 400 fold to achieve the starting concentration for each experiment. Appropriate controls including negative droplet and no template controls can be included. The iScript™ Reverse Transcription (RT) Supermix, 100×20 μl reactions (Bio-Rad: 170-8841) can be diluted to 1× with corresponding volumes transferred to each reaction. 50 μL reaction mixtures containing RT mix, primers, template and QX200™ ddPCR EvaGreen Supermix (Bio-Rad: 186-4034) can be divided in to 20 μL each. Droplet generation and transfer of emulsified samples to PCR plates can be performed according to manufacturer's instructions (Instruction Manual, QX200™ Droplet Generator—Bio-Rad). The cycling protocol follow with a 95° C. enzyme activation step for 5 minutes followed by 40 cycles of a two-step cycling protocol (95° C. for 30 seconds and 60° C. for 1 minute). The ramp rate between these steps can be 2° C./second. Post-cycling protocol can be in accordance with the kit instructions (Bio-Rad—186-4034). The absolute quantity of DNA per sample (copies/μL) can be processed using QuantaSoft (v. 1.7.4).

### 2. Example 2: Define the Presence of a Novel Protein Product from EGFR_PR20CTF and Examine Signaling of EGFR_PR20CTF to Develop Drug Repurposing Strategies for ccRCC

The hypothesis that EGFR_PR20CTF generates a novel truncated protein in ccRCC tumors leading to downstream kinase cascade signaling activation independent of ligand and promoting carcinogenesis and progression can be tested. First, that EGFR_PR20CTF is expressed at the protein level can be tested in ccRCC in cell lines and tumor tissues. Initial experiments use immunoblotting for EGFR with an antibody that targets the C-terminal region/kinase domain (Abcam ab32077, Recombinant Anti-EGFR antibody [E235] directed at residues 1150-1210), which can detect the full-length protein and the splice variant, separated by MW using sodium dodecyl sulfate-polyacrylamide gel electrophoresis (SDS-PAGE). EGFR_PR20CTF contains the last 386 amino acids, so it is expected to migrate at ˜44 kDa. As a control, immunoblots for the same set of samples with an antibody to the N-terminal portion of the protein (Cell Signaling #2256, anti-EGFR Mouse monoclonal antibody (mAb)) targeting the extracellular domain and rated for immunoprecipitation only detects the full-length sequence.

To detect the specific N-terminal peptide of EGFR_PR20CTF, this experiment need to be supplemented with immunoprecipitation, in-gel digestion, and liquid chromatography-tandem mass spectrometry (LC-MS/MS) peptide sequencing (using well-established methods), given that the antibody can detect processed forms, truncations, or posttranslational cleavage products. Cell lines with endogenous expression or overexpression of this splice variant can be used to immunoprecipitate all EGFRs with an antibody targeting the C-terminal portion of the protein (Abcam ab32077) and separate the full-length protein from the splice variant with SDS-PAGE. After Coomassie staining, the region that contains the lower molecular weight (MW) EGFR_PR20CTF proteoform can be excised and digest the proteins with trypsin before LC-MS/MS peptide sequencing. The peptide indicative of the splice variant of interest is MNYLEDR (or MNYLEDRR if there is not full tryptic cleavage of the peptide); GMNYLEDR can be observed for the full sequence. In addition to using data-dependent mass spectrometry, a parallel reaction monitoring (PRM) scan can be programed in a specific attempt to detect that N-terminal peptide from EGFR_PR20CTF, which can be evidence that the protein construct is present as predicted. These experiments are also performed on tumor-tissue homogenates to determine the extent to which this proteoform is expressed in human tissues. To determine the expression level of each proteoform, the quantification of levels of full-length WT EGFR and EGFR_PR20CTF can also be performed using SDS-PAGE and targeted proteomics.

To test whether EGFR_PR20CTF can function as a kinase, this protein product (Rabbit Reticulocyte Transcription and Translation System, Promega) can be expressed and use it for in vitro kinase assays with peptides containing EGFR autophosphorylation sites and sequences of other known EGFR targets. Mass spectrometry-detected unmodified and phosphorylated peptides can be used to confirm whether this protein is a functional kinase. If the EGFR_PR20CTF protein does function as a kinase, the in vitro kinase assay can be used to determine whether small molecule inhibitors of EGFR (e.g. erlotinib, gefitinib, or afatinib) can inhibit this proteoform. If EGFR_PR20CTF does not function alone as a kinase, the variant proteoform can be reconstituted with full-length WT EGFR (Origene TP710011) and compare kinase assays of each proteoform alone to the mixture of proteoforms. Additional testing can also be performed to see whether the mixture of WT EGFR and EGFR_PR20CTF is sensitive to small molecule inhibitors.

In case the initiator methionine is removed, an alternative strategy to detect EGFR_PR20CTF protein is to use PRM to look for NYLEDR/NYLEDRR. If these peptides are not detected, the remaining sequence can be examined and perform semitryptic database searches to locate another N-terminal sequence for the EGFR construct observed at lower MW. Antibodies targeting tyrosine 1068 phosphorylation (e.g. Abcam ab52894, Anti-EGFR antibody [EP38Y]) can also be used to explore whether the EGFR-PR20CTF protein is phosphorylated. Activity-based protein profiling (ABPP) with desthiobiotinylating probes (ActivX, Thermo Fisher) can also be used to determine whether EGFR_PR20CTF can bind adenosine triphosphate (ATP). In WT EGFR, K745 is the site modified by ABPP when ATP is bound to the active site; lysines near autophosphorylation sites are also labeled by proximity.
a) Probing Interactions Between EGFR-PR20CTF and Full-Length WT EGFR:

Investigation of the hypothesis that EGFR_PR20CTF interacts with full-length EGFR. The last 386 amino acids of EGFR contain the sequence 825-1210, which includes all of the known autophosphorylation sites. However, the dimerization domain (688-704) is not included in the sequence, and the kinase domain (712-979 in the full-length protein) is not intact. Specifically, some residues for ATP binding are missing, but the active site residue, D837, and one of the ATP binding contacts, D855, are still present. Therefore, it is unclear whether this proteoform interacts with full-length EGFR or generate its own signaling cascade unrelated to full-length EGFR. Full-length EGFR is immunoprecipitated using an antibody that recognizes the N-terminal region of the protein (Cell Signaling #2256, anti-EGFR Mouse mAb rated for IP), which were used for IP-LC-MS/MS analysis of EGFR phosphorylation. To confirm protein-protein interactions and detect the peptide specific for the splice variant, separation by SDS-PAGE, immunoblotting with the C-terminal EGFR antibody, and analysis of digested proteins from lower MW gel bands with LC-MS/MS and liquid chromatography-parallel reaction monitoring mass spectrometry can all be preformed.

To further determine whether EGFR_PR20CTF functions similarly to full-length WT EGFR, IP and immunoblots can also be used. This allows for the ability to see whether EGFR_PR20CTF can bind to adapter proteins, such as GRB2, to promote downstream signaling. Another alternative strategy is to investigate the hypothesis that EGFR_PR20CTF forms protein complexes that do not include full-length EGFR. For this BioID or APEX experiments can be conducted to compare the interacting proteins of the full-length EGFR and the EGFR splice variant. Fusion proteins are made for EGFR and EGFR_PR20CTF, which produce a reactive biotinylation cloud around EGFR_PR20CTF, thereby labeling adjacent and binding proteins. A comparison of the full-length and splice variant protein complexes detected in LC-MS/MS protein identification experiments can be used to determine whether these EGFR proteoforms participate in similar or different complexes.

b) Elucidation of EGFR_PR20CTF Signaling.

The hypothesis that EGFR_PR20CTF interacts with full-length EGFR to enhance EGFR signaling can be investigated and then examined whether this signaling is independent of EGF stimulation. We have identified relevant ccRCC cell lines through Cancer Cell Line Encyclopedia (Table 2).

TABLE 2
Proposed ccRCC cell lines that demonstrate positive
(A-498, 786-O) and negative (CAL-54, 769-P)
expression of the α-EGFR_PR20CTF splice variant.

| | Known EGFR | EGFR_PR20CTF | EGFR_PR20CTF |
|---|---|---|---|
| Cell Line | Reads | Reads | Percent |
| A-498 | 301 | 28 | 8.46 |
| 786-O | 164 | 7 | 4.07 |
| CAL-54 | 453 | 0 | 0 |
| 769-P | 294 | 0 | 0 |

Abbreviations:
ccRCC, clear cell renal cell carcinoma;
EGFR, epidermal growth factor receptor.

Targeted proteomics and immunoblotting using cell line models with endogenous PGP-21 expression, overexpression, and knockout/knockdown of the EGFR splice variant allows for the confirmation of the expression levels of full-length and splice variant EGFR. After verifying the expression levels of EGFR and EGFR_PR20CTF, phosphoproteomics can be performed to identify differences in signaling. These experiments can be conducted using immunoprecipitation of tyrosine phosphorylated peptides (Cell Signaling Technologies PTMscan Kits) and basic pH-reversed phase liquid chromatography for peptide fractionation prior to enrichment of phosphopeptides by immobilized metal affinity chromatography (IMAC) (Cell Signaling Technology 20432, IMAC Fe-NTA Magnetic Beads).

Additional experiments with dose escalation or time course studies of EGF show whether the splice variant overcomes the need for EGF stimulation. In addition to these experiments, the phenotypes associated with overexpression and knockdown of EGFR_PR20CTF can be examined by using colony formation assays, including Matrigel invasion, to see whether this protein construct plays a role in metastasis and poor patient outcomes. When experiments show that EGFR_PR20CTF initiates the same signaling cascades as full-length WT EGFR, the hypothesis that EGFR_PR20CTF can rescue cells after loss of full-length EGFR can be explored. Viability can be assessed after EGFR CRISPR knockout with and without EGFR_PR20CTF expression. Phosphoproteomics (as described above) can be used to determine whether EGFR signaling is reconstituted or if novel signaling pathways are activated by EGFR_PR20CTF. Modulation of key signaling hubs can be verified by immunoblotting.

c) Inhibition of EGFR_PR20CTF Signaling in ccRCC Cell Lines.

The hypothesis that signaling from the EGFR splice variant can be targeted with existing EGFR kinase inhibitors to eliminate the aggressive phenotype observed in the clinic can be tested. Experiments in this section can be carried out in parallel with the experiments proposed in the above section, Elucidation of EGFR_PR20CTF Signaling. Phosphoproteomics and ABPP can be used to examine changes in signaling with EGFR inhibitors (eg, erlotinib, gefitinib, or afatinib) in ccRCC cell lines that express full-length WT EGFR and EGFR_PR20CTF. Therapeutic mAbs (eg, cetuximab) can be used to isolate the signal changes due to the full-length EGFR. In addition, CRISPR for full-length or splice variant EGFR can also be used to identify specific effects of each EGFR proteoform.

d) Data and Statistical Analysis Plan

Herein the presence of EGFR_PR20CTF variants in 77 tumor samples collected is identified and characterized. All statistically analyzed data can be conformed to a normal distribution as determined using a Shapiro-Wilk test. A Student's t-test or Mann-Whitney test can then be used to assess the statistical significance between the two-fold dilutions of DNA for each sample. The % coefficient of variation (standard deviation/mean*100) can be used to assess inter-sample variability. To determine prevalence of EGFR_PR20CTF expression in metastatic tumors using core biopsy samples frequency tables can be generated and tabulated number of successful samples that undergo RNA extraction and ddPCR. Results can be compared using Chi-square homogeneity test between samples from primary and metastatic samples along with those from surgical resections vs. biopsies.

To identify associations for tumor samples that harbor the EGFR_PR20CTF splice forms, the alpha variant, patients are separated in to four ordinal groups based on tertiles of their expression levels of the variants. The groups can be stratified as follows; Group 0=no EGFR_PR20CTF expression; Tertile-1=<1-33% of normalized EGFR_PR20CTF expression (lowest third); Tertile-2=33−66% expression (middle third); Tertile-3=>66% expression of maximum value (highest third). The basis of this stratification is to maximize the ability to assess the direction and magnitude of clinically relevant associations with the levels of EGFR_PR20CTF splice forms because there is currently no biological or clinically relevant cutoff to determine significance of the variants. Association analysis can be performed using the splice variant as a quantitative variable. The survival data with a survival event rate of 37% and percentages of EGFR splice variant with a standard deviation of 0.081, a total sample size of 69 evaluable subjects achieves at least 90% power at a significance level of 5% to detect an expected log risk (hazard) ratio of 7.92 using a Cox proportional hazard regression. Considering a dropout rate of 10%, at least 77 subjects can be accrued in the prospective cohort. Also include can be retrospective patients who satisfy the inclusion and exclusion requirements, further adding to the power of this analysis.

As a primary analysis associations can be evaluated with primary treatment resistance to immunotherapy based regimens. Primary resistance can be defined as PFS≤15 weeks necessitating a change in treatment regimen for patients with metastatic ccRCC receiving first-line treatment with immunotherapy-based treatment regimens. The most common RNA-seq-driven EGFR splice variant, c.2470-188, a stratified Cox regression analysis of PFS and overall survival (OS) after immunotherapy can be performed. For the secondary objectives, EGFR_PR20CTF expression can be correlated with OS and radiologic response, using descriptive analysis with scatter plots and regression analysis can all be focused on. Specific drug regimens (i.e. Ipilimumab and Nivolumab, Pembrolizumab and Axitinib, etc.) can be adjusted for with known predictors like The International Metastatic Renal Cell Carcinoma (IDMC) risk group stratification, as part of a multivariable analysis. Square root or logarithmic transformation of the data can be used to improve normality of the distributions (determined as the transformation (including untransformed) with the lowest Anderson-Darling normality score). Secondary resistance to immunotherapy regimens in the first line setting defined as PFS<30 weeks necessitating a change in treatment regimen can be investigated and analysis can be conducted in a similar fashion that perform in the primary objective. In addition, Kruskal-Wallis test can be performed to assess the association of EGFR_PR20CTF expression with an objective response outcome using the iRECIST criteria. Drug toxicities may have played a role in the data showing poor benefits of immunotherapy and can conveniently be captured in the prospective cohort. The presence and rate of dose limiting toxicities can be analyzed using a multivariable logistic regression with specific drug and IDMC risk group as covariates as well as demographic covariates. All toxicity data can be summarized by category and grade in a standard fashion. Proportions experiencing the most common types of toxicity in this trial can be estimated and 95% confidence intervals calculated based on the Pearson and Clopper method for binomial proportions can be reported. The category and grade of toxicity can then be stratified by the level of EGFR_PR20CTF expression to look for possible associations which are important variables in decisions to stop immunotherapy based regimens and possible lead to decreased overall survival.

### 3. Example 3: Aberrant Growth Factor Receptor RNA Splice Products are Among the Most Frequent Somatic Alternations and are Associate with Poor Response to Immunotherapy

a) Results

(1) Demographics and Characterization of EGFR_pr20CTF in Renal Cell Cancer Tumors.

In this study, a recently identified novel epidermal growth factor receptor (EGFR) aberrant splicing variant is described that is unique to clear cell renal cell carcinoma (ccRCC) and present in up to 50% to 60% of all patients in the multi-institutional cohort of patients with a range of local and metastatic disease. The aberrant EGFR_PR20CTF in ccRCC is shown in FIG. 1. Three different splice forms were observed, which were labeled as alpha, beta, and gamma on the basis of their increasing distances upstream from the exon 21 50 binding site. The α-isoform is the predominate isoform, occurring in >95% all tumors that demonstrate EGFR_PR20CTF. The distribution of the EGFR_pr20CTF iso-forms across is detailed in FIG. 2.

This novel aberrant splice variant resides within EGFR, which has been confirmed to be stably and endogenously expressed in selected ccRCC cell lines and human tumors. It is predicted to result in a novel C-terminus fragment, beginning between exons 20 and 21, which was labeled aberrant EGFR splice variant (EGFR_PR20CTF). This novel aberrant isoform is preferentially expressed in patients with advanced and metastatic (vs local/indolent) disease and is found in >72% of patients with >pT3 disease and in 90% of metastatic tumor samples.

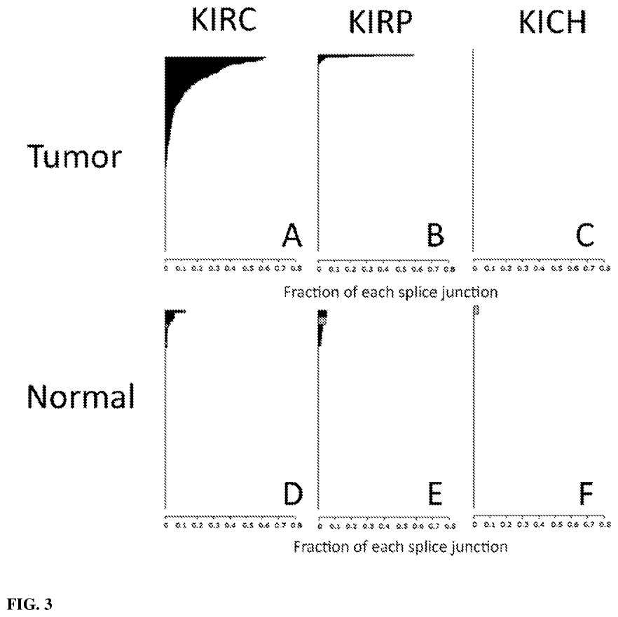

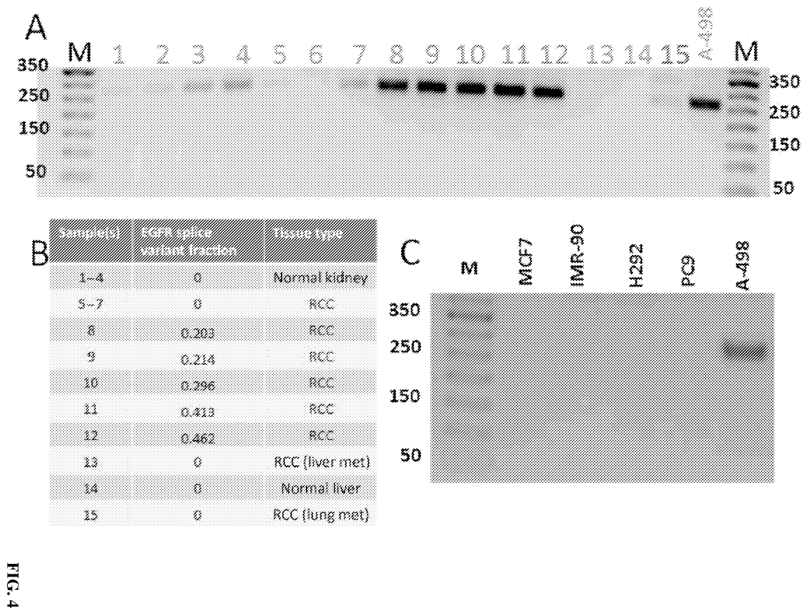

To assess the frequency of EGFR_PR20CTF in ccRCC, a cross-sectional analysis was performed using RNA sequencing (RNA-seq) data. One or more reads demonstrating EGFR_PR20CTF were identified in 76.1% (n=67/88) and 56.7% (n=279/492) of tumors from the institutional high-risk ccRCC and The Cancer Genome Atlas Kidney Renal Clear Cell Carcinoma (TCGA-KIRC); institutional cohorts, respectively)(FIG. 3). EGFR_PR20CTF alterations in both the institutional ccRCC and KIRC cohorts were more common than many of the well-described somatic alterations seen in ccRCC cases, including PBRM1, SETD2, and KDM5C. We compared the frequency of EGFR_pr20CTF with other commonly mutated genes in ccRCC by evaluating KIRC samples with available matching WES (n=420/492). In this subcohort of 420 tumors, the frequency of EGFR_pr20CTF was 58.1% (n=244), which makes it a more common alteration than many well-defined somatic ccRCC mutations. The high frequency of the aberrant EGFR_PR20CTF splicing event observed in ccRCC makes EGFR_PR20CTF an exciting target, as investigations into this alteration can yield results with broad applicability as a biomarker and potential treatment target.

(2) Specificity of EGFR_pr20CTF

Although EGFR activation occurs in many cancers, the exact mechanism by which it is activated is highly disease dependent and can occur via amplification (i.e., breast cancer), activation of mutations and deletions of the kinase domain (i.e., lung cancer) or inactivating deletions of the extracellular domain (i.e., glioblastoma). EGFR_PR20CTF splice forms do not include 5′ splicing events, which indicates a novel transcription initiation event at exon 20. Also, the amino acid in exon 21 is a methionine, indicating that this EGFR_PR20CTF is the beginning of a novel transcript from which a protein product can arise. This isoform can contain the 386 C-terminal amino acids, including much of the kinase domain, but can have a loss of the extracellular and transmembrane components of the normal protein product. These novel isoforms can allow for the development of screening tests (blood/urine) to yield biomarkers of disease or the development of targeted agents specific to this product (small molecule inhibitors or chimeric antigen receptor T (CAR-T) cells/antibodies).

The high specificity of EGFR_PR20CTF was validated in ccRCC by analyzing a cohort of 606 non-RCC tumors with RNA-seq from the institutional Total Cancer Care (TCC) cohort and 491 consecutive samples (tumor and normal) from TCGA (GBM, COAD, READ, STAD, LUSC, and LUAD), with 12 different cancer types being represented. The presence of EGFR_PR20CTF in <0.2% of non-RCC samples (n=2/1091); both of these samples had very low levels of gene expression.

(3) Validation of EGFR_pr20CTF

Using semiquantitative reverse transcription-polymerase chain reaction (RT-PCR), the PCR product was validated for the α-EGFR_PR20CTF splicing event in 5 tumor samples and in the RCC cell line A-498, which expressed the variant via RNA-seq (FIG. 4). Negative EGFR_PR20CTF ccRCC tumor tissue and adjacent normal kidney tissue (no tumor) were also included as controls. The presence of low levels of EGFR_PR20CTF in a few normal adjacent tissues is interesting, and similarly low levels in normal tissue samples (18.8% [n=13/69]) from TCGA KIRC. The expression levels in these samples were much lower, which can be explained by tumor contamination of these tissue samples.

(4) Clinical Associations

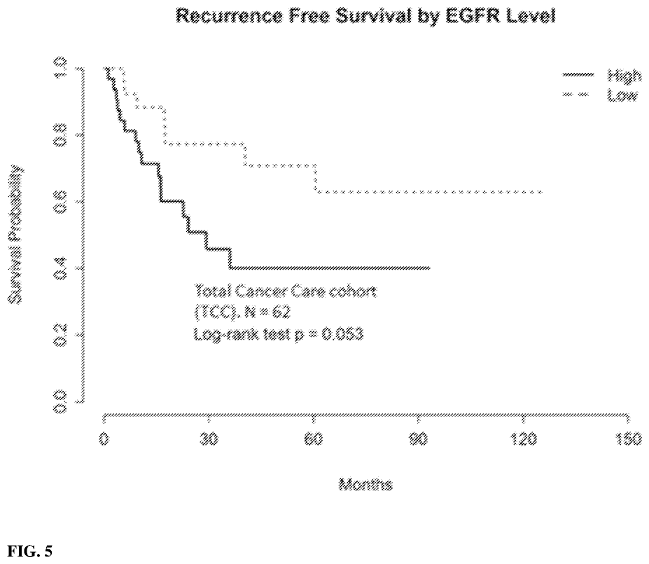

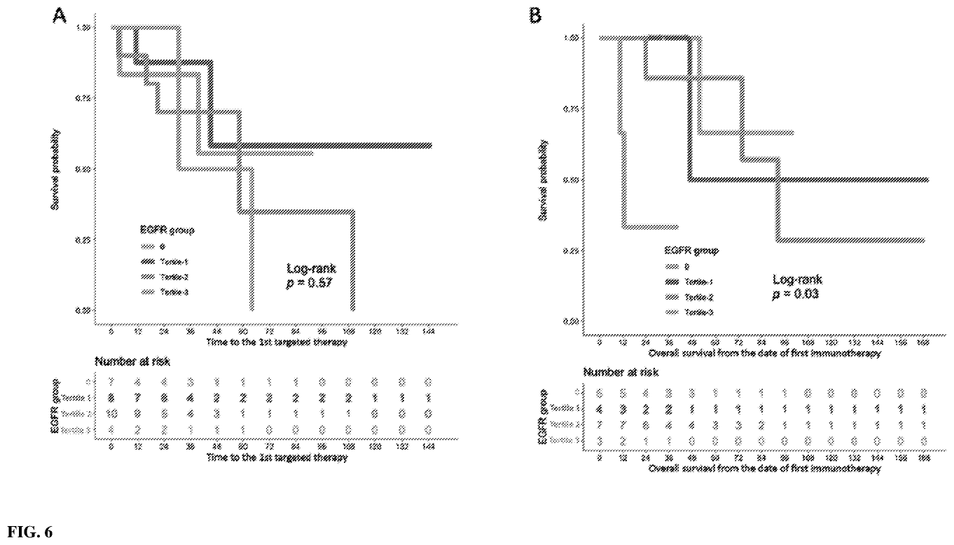

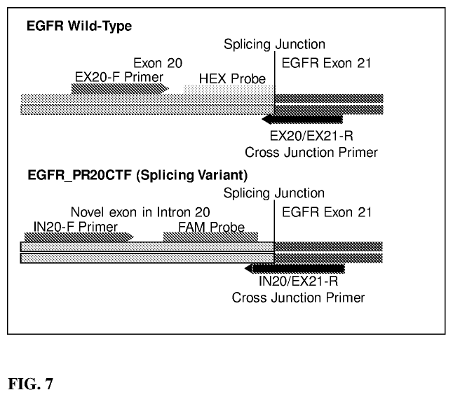

To determine the possible clinical impact of EGFR_PR20CTF splice forms in ccRCC the presence of the splice variant and associations of cancer recurrence and response to immunotherapy treatment was examined, FIGS. 5 and 6. In order to analyze response of immunotherapy to those patient with metastatic ccRCC patients who showed expression of the variant by RNA-seq were grouped into tertiles based on the levels of EGFR_pr20CTF expression; placing patients in either the lowest, middle or highest tertile and compared them to patients with no expression of the variant, Group 0. We measured time to death (months) following first systemic therapy treatment, which included interleukin-2, atezolizumab, nivolumab, pembrolizumab, ipilimumab, cabozantinib, axitinib, pazo-panib, sorafenib, sunitinib, levatinib, or everolimus. Patients with the highest expression of the EGFR_pr20CTFsplice variant had significantly lower survival at 48 mo following immunotherapy regimens compared with patients with the lowest expression of EGFR_pr20CTF (p=0.036; FIG. 6). The average survival in patients with high EGFR_pr20CTF expression was <16 mo. Among 19 eligible patients who received immunotherapy (interlukiun-2 (IL-2) or Checkpoint inhibitor), a statistically significant difference in survival from the time of their first immunotherapy treatment was found.

b) Discussion

In this study, we identified and characterized the previously unrecognized alternative 50 start site within intron 20 of EGFR, which gives rise to aberrant splice variants in RCC. These variants were found to be among the most frequent molecular alterations in ccRCC. At least one read demonstrating EGFR_pr20CTF was identified in 76.1% (n=67/88) and 56.7% (n=279/492) of tumors in an institutional RCC and the TCGA KIRC cohorts, respectively. EGFR_pr20CTF alterations in the ccRCC and KIRC cohorts were more com-mon than many of the well-described somatic alterations seen in ccRCC, including PBRM1, SETD2, KDM5C, and even VHL

These splice variants were highly specific to RCC. Although traces of EGFR_pr20CTF were found in non-ccRCC tumors, levels were much lower than what were seen in ccRCC and relatively infrequent. The presence of EGFR_pr20CTF was seen in a few normal adjacent tissues in the TCGA cohort analysis and in the RT-PCR studies. This is an interesting finding and should be further evaluated, but it could be explained by tumor contamination. These infrequent examples occurred at much lower levels and were primarily among the ccRCC samples. Finding of EGFR_pr20CTF in other solid tumors was extremely rare.

We observed that EGFR_pr20CTF splice forms do not include 50 splicing events, which suggests a novel transcription initiation site within intron 20. In addition, a methionine in exon 21 indicates that this EGFR_pr20CTF is the beginning of a novel transcript from which a protein product could arise. This isoform contains the 386 C-terminal amino acids, including some from the kinase domain.

In this study, we saw a higher frequency of EGFR_pr20CTF in the ccRCC TCC tumors than in the KIRC dataset. This could be due to the differences in the patient population, as evidenced by the percentage of patients with stage pT3/T4 tumors in each cohort (TCC ccRCC=72.7% [n=64/88]; KIRC=35.8% [n=193/420]). Furthermore, we included specimens from metastatic tumors in the cohort, which could explain the enrichment of EGFR_pr20CTF that was seen in the population; 90% (9/10) of metastatic tissue samples demonstrated EGFR_pr20CTF. The differences would also seem to suggest that the development of these EGFR variants is a subclonal event in the evolution of the tumors to a more aggressive clinical phenotype.

c) Methods

(1) EGFR_PR20CTF Detection

(a) Primers and their Design Strategy

Based on the RNA sequence of the aberrant splice variant, we have designed a set of primers and probes that specifically amplify and quantify the novel splice variant EGFR_PR20CTF including an alternative primer set (Table 4).

TABLE 4
Sequences for primers and probes

| Primer/probe | Sequence (5→3) | Size | Tm | Note |
|---|---|---|---|---|
| EX20-F | TATTGGCTCCCAGTACCTGC | 61 bp | 55.5 | forward primer for wildtype |
| | (SEQ ID NO: 1) | | | |
| EX20/EX21-R | CAAGTAGTTCATGCCCTTTGC | | 54.1 | reverse bridging primer for wild type |
| | (SEQ ID NO: 2) | | | splice |
| EX20-P | TGGTGTGT+GCA+GATCGCAAAGG | | 63.6 | +before a base means it is LNA. |
| (probe) | (SEQ ID NO: 3) | | | Label the probe with VIC or HEX |
| | | | | (wildtype probe) |
| IN20-F | CATATACGAGCACCTCTGGACTT | 73 bp | 55.7 | forward primer for novel splicing |
| | (SEQ ID NO: 4) | | | exon |
| IN20/EX21-R | CCAAGTAGTTCATGCCCATTA | | 51.9 | reverse bridging primer for alt splice |
| | (SEQ ID NO: 5) | | | point |
| IN20-P | GGAA+CT+GGAT+GGA+GAA+AAGTT | | 62.5 | +before a base means it is LNA. |
| (probe) | (SEQ ID NO: 6) | | | Label the probe with FAM (splicing |
| | | | | probe) |
| Alternative | | | | |
| primer set | | | | |
| EX20-F-a | TATTGGCTCCCAGTACCTGC | 68 bp | 62.8 | forward primer for wildtype |
| | (SEQ ID NO: 7) | | | |
| EX21-R-a | GGTCCTCCAAGTAGTTCATGC | | 61.6 | reverse common primer |
| | (SEQ ID NO: 8) | | | |
| IN20-F-a | CATATACGAGCACCTCTGGACTT | | 62.8 | forward primer for novel splicing |
| | (SEQ ID NO: 9) | | | exon |
| EX21-R-a | GGTCCTCCAAGTAGTTCATGC | 79 bp | 63 | reverse common primer |
| | (SEQ ID NO: 10) | | | |

Based on the RNA sequences of the candidate splice variants, we have designed a set of primers and probes that specifically amplify and quantify each novel splice variant (Table 1). To increase specificity, one primer was designed across the splicing junction. Considering RNA degradation in blood, the amplicon size is limited to <70 bp. As an example, for EGFR splice variant detection, a forward primer was designed in the novel exon of the splice variant (part of EGFR intron 20), FIG. 7 The reverse primer targeted the junction region between the novel exon and EGFR exon 21. In the case of EGFR, we also designed primers for wild-type EGFR detection for direct comparison with the splice variant. A forward primer was designed within exon 20 and a reverse primer targeted exon 20/21 junction. To ensure their compatibility for multiplex dPCR, all primer pairs have an annealing temperature of 60° C. All four dyes can be excited at a single wavelength (488 nm) but emit at distinctly different wavelengths. The splice variant probes are labeled with FAM, JOE and ROX for EGFR, PDZD2 and FAM107B, respectively. Wildtype EGFR is labeled with TAMRA.

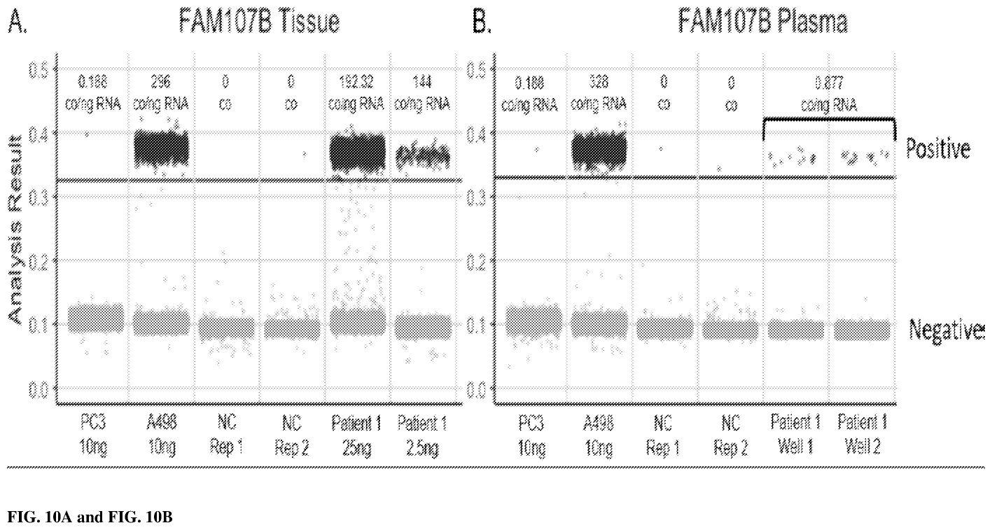

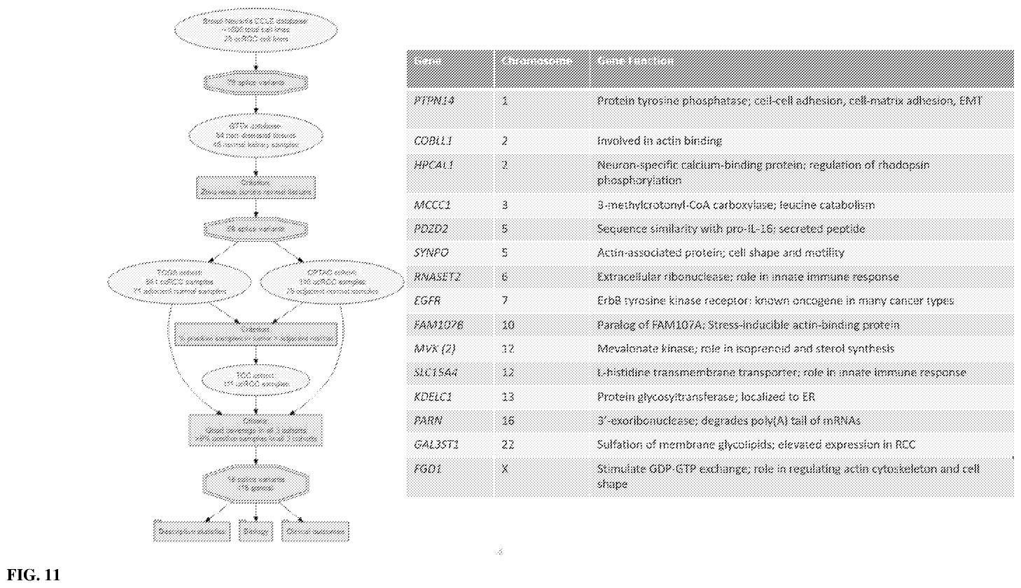

We have tested the multiplex assay using QIAcuity Digital PCR System (Qiagen). This system allows for 26000 partitions in one reaction and detects five separate fluorescent dyes simultaneously. Our initial testing in both cell line RNAs, tumor and plasma cfRNAs showed quantitative detection of these targets in the multiplex assay (FIG. 10).

(b) Exosome Isolation and RNA Extraction

The ExoQuick ULTRA kit (System Biosciences) is used for exosome isolation. Specifically, 1 ml of platelet-poor plasma is centrifuged at 10 000 g at 4° C. for 10 minutes. The resultant supernatant is mixed with appropriate amount of ExoQuick reagent and then centrifuged at 3000 g for 10 minutes. The pellet is further purified by going through a specialized column to collect the exosome fraction. Total RNA is extracted using the miRNeasy Micro Kit (Qiagen) and quantified using the Bioanalyzer Small RNA Analysis kit (Agilent). As a comparison, total RNA is also extracted from 1 ml of platelet-poor plasma from the same patient.

(c) ddPCR Assay

Detection of EGFR_PR20CTF in patients' plasma is performed in a ddPCR instrument. For EGFR_PR20CTF, a previously designed TaqMan real-time PCR assay for EGFR_PR20CTF and wild-type EGFR is used (Table 4). Amplicon size is 73 bp for EGFR_PR20CTF and 61 bp for wild-type EGFR. For both the EGFR_PR20CTF assay and other ideal candidate splice variant assays, cDNA is first made by using the iScript Reverse Transcription Supermix (Bio-Rad Laboratories). ddPCR is performed in the reaction mixtures containing cDNA, primers/probe mix, and QX200 ddPCR™ Supermix for Probes (Bio-Rad Laboratories). The cycling protocol follows with a 95° C. enzyme activation step for 5 minutes followed by 40 cycles of a two-step cycling protocol (95° C. for 30 seconds and 60° C. for 1 minute). The absolute quantity of target RNA per sample (copies/μL) is processed using QuantaSoft (v. 1.7.4).

(2) Clinical Samples and Bioinformatics

To further investigate the frequency of this alternative splicing event, we identified 699 patients whose tumors had undergone whole-exome sequencing (WES), RNAseq of tumor, and germline DNA sequencing. Of these, 99 specimens were collected from RCC tumors (89 primary and 10 metastatic tumors). Using RNAseq techniques, we aligned the institutional high-risk cohort with a human reference genome to detect the frequency of EGFR_pr20CTF alternate splicing events.

(3) The Cancer Genome Atlas Analyses

To assess the frequency and specificity of EGFR_pr20CTF in a larger cohort of patients, we analyzed the RNAseq from The Cancer Genome Atlas (TCGA). We identified 492 patients with kidney renal clear cell carcinoma (KIRC), 190 with papillary RCC, and 50 with chromophobe RCC. We also evaluated adjacent normal tissue samples and a subset of non-RCC tumors from TCGA.

(4) Gene Expression (Reverse Transcriptase-Polymerase Chain Reaction) in Tumor Tissues and Cell Lines

To validate the presence of EGFR_pr20CTF in RCC, we performed reverse transcriptase-polymerase chain reaction (RT-PCR) using primers amplifying the novel EGFR_pr20CTF splice isoform junction in tumor tissues and a representative human RCC cell line. RNA was isolated and purified from tumor tissue and A-498 (HTB-44; ATCC, Manassas, VA, USA) cells as per the manufacturer's instructions. Next, RNA was reverse transcribed into cDNA, which was then used to perform PCR using primers designed to target intron 20 and exon 21 of EGFR. The PCR products were visualized on agarose gel using gel electrophoresis. Normalization was performed relative to beta-actin.

### 4. Example 4: Novel Splice Variants in ccRcc

The current understanding of actionable genomic mutations in clear cell renal cell carcinoma (ccRCC), the most common type of kidney cancer, and their role in managing ccRCC remains sparse compared to that of other malignancies. In the majority of patient interactions, specific mutations in ccRCC and the disease's mutational burden have not proven to be clinically impactful. Despite this focus on mutational burden, diagnostic, prognostic, and therapeutic targets for kidney cancer are relatively nonexistent. Compared to mutational alterations in ccRCC, the study of alternative and aberrant mRNA splicing as potential biomarkers, drivers, and therapeutic targets of kidney cancer is limited. We first identified, a novel epidermal growth factor receptor (EGFR) aberrant splicing variant that is relatively specific to ccRCC and present in both primary and metastatic tumor samples. This effort led to the investigation of other splice variants more comprehensively among ccRCC tumors. We identified these splice variants by examining RNA-seq data from 935 cell lines from the Cancer Cell Line Encyclopedia (CCLE). Using Portcullis, we then analyzed an institutional Total Cancer Care (TCC) database for variants with highly prevalent in ccRCC. The TCC database includes more than 4,000 sample from 27 different tumor types. In this analysis, we refined the recognition of positive samples by limiting detection to samples that had at least 1% of reads mapping to the novel splice variant to minimize artifacts. We then validated these findings using ccRCC tumors and adjacent normal samples from Clinical Proteomic Tumor Analysis Consortium (CPTAC), The Cancer Genome Atlas (TCGA) and Genotype-Tissue Expression Project (GTEx). The goal was to identify splice variants that we believe are excellent candidates for biomarker development based on their frequency and relatively specificity for ccRCC. Across all three ccRCC cohorts we found at least this work identified three candidate splice variants, Family With Sequence Similarity 107 Member B (FAM107B), PDZ Domain Containing 2 (PDZD2) and EGFR are ideally suited for evaluation as a liquid biomarker among ccRCC patients, Table 1. At least one of these variants is present in >95% of patients across all three ccRCC cohorts. Additionally, we can employ alternative primer sets for each splice variant as needed and consider new splice variants targets, Table 3. Since the splice variants are relatively rare in circulating fluids, false negative results are more likely from low cfRNA input, so when possible, we can start with larger plasma or urine volume to increase yield of cfRNA.

TABLE 1
Detection frequency of candidate aberrant splice variants (SV) among clear cell renal cell carcinoma
(ccRCC) tumors from The Cancer Genome Atlas (TCGA), Clinical Proteomic Tumor Analysis Consortium (CPTAC)
and Total Cancer care (TCC). We also examined available samples from non-ccRCC tumor in TCC and adjacent
normal samples from CPTAC and TCGA. Samples were considered positive if they had at least 1% reads
across the novel SV, normalized by dividing all junction reads overlapped with the SV.

| | | | | | | | Normal | Non-RCC |
|---|---|---|---|---|---|---|---|---|
| | | Clear Cell RCC | | | Adjacent Normal Kidney | | Kidney | Tumors |
| Splice | | TCGA | CPTAC | TCC | TCGA | CPTAC | GTEx † | TCC |
| Variant | SV location* | (N = 491) | (N = 110) | (n = 111) | (N = 71) | (N = 75) | (n = 45) | (n = 4,174) |
| FAM107B | 10:14664173-14709632 | 461 (94%) | 82 (75%) | 99 (89%) | 19 (27%) | 19 (25%) | 0 (0%) | 81 (2%) |
| PDZD2 | 5:31995744-32000244 | 248 (51%) | 85 (77%) | 95 (85%) | 16 (23%) | 50 (67%) | 0 (0%) | 1235 (30%) |
| EGFR | 7:55259222-55259411 | 248 (51%) | 69 (63%) | 54 (49%) | 10 (14%) | 27 (36%) | 0 (0%) | 125 (3%) |

† Samples from GTEx (The Genotype-Tissue Expression Project) were considered positive it greater than one read mapped to the novel SV
*= Genecode V19, based on GRch37.

TABLE 3
Table 3. Detection frequency of alternate aberrant splice variants (SV) among clear cell renal cell carcinoma
(ccRCC) tumors from The Cancer Genome Allas (TCGA), Clinical Proteomic Tumor Analysis Consortium (CPTAC)
and Total Cancer care (TCC). We also examined available samples from non-ccRCC tumor in TCC and adjacent
normal samples from CPTAC and TCGA. Samples were considered positive if they had at least 1% reads across
the novel SV, normalized by dividing all junction reads overlapped with the SV.

| | | | | | | | Normal | Non-RCC |
|---|---|---|---|---|---|---|---|---|
| | | Clear Cell RCC | | | Adjacent Normal Kidney | | Tissue | Tumors |
| Splice | | TCGA | CPTAC | TCC | TCGA | CPTAC | GTEx † | TCC |
| Variant | SV location* | (N = 491) | (N = 110) | (n = 111) | (N = 71) | (N = 75) | (n = 11,688) | (n = 4,174) |
| MVK | 12:110032708-110032832 | 443 (90%) | 103 (94%) | 100 (90%) | 35 (49%) | 68 (90%) | 0 (0%) | 125 (3%) |
| SEC14L6 | 22:30927985-30928166 | 118 (24%) | 77 (70%) | 71 (64%) | 28 (40%) | 19 (25%) | 0 (0%) | 250 (6%) |
| HPCAL1 | 2:10529703-1053696 | 463 (94%) | 100 (91%) | 89 (80%) | 10 (14%) | 27 (36%) | 0 (0%) | 138 (3%) |

† Samples from GTEx (The Genotype-Tissue Expression Project) were considered positive it greater than one read mapped to the novel SV. Samples from GTEx constituted 30 different organ and tissue types from non-cancer patients.
*= Genecode V19, based on GRch37.

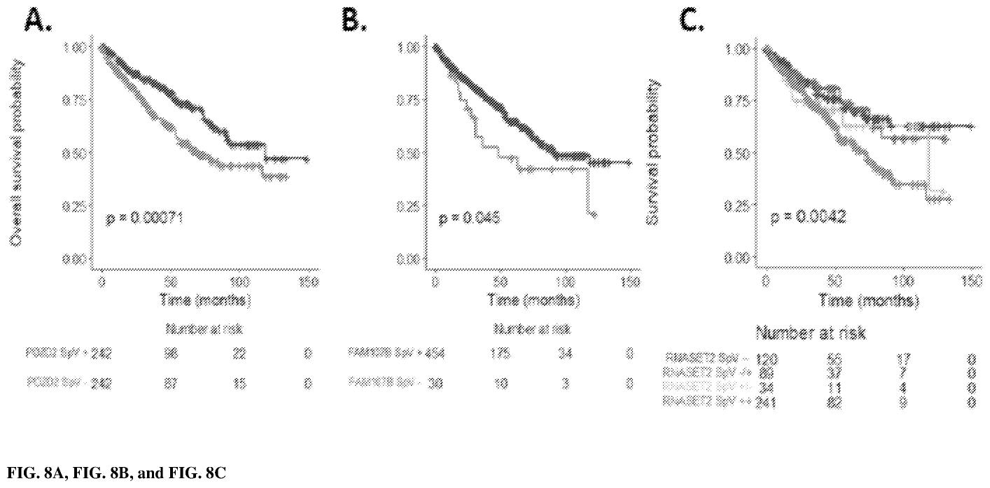

While the primary goals were to identify splice variants for investigation as a possible liquid biomarker, we did examine the clinical associations of these variants with clinical outcomes across the three ccRCC cohorts. Within the largest cohort with adequate follow up data, TCGA (KIRC), patients without detection of FAM107B and PDZD2 we found to have inferior overall survival, p-value=0.04 and <0.01 respectively, FIG. 8. A similar trend was seen for EGFR but this did not meet significance (p-value=0.06). Among the TCC cohort we continued to see a significance of inferior survival among patients without PDZD2, p-value=<0.01. CPTAC had limited survival data for analysis. Table 5 shows the primers and amplicons used for PDZD2, FAM107B and HPCAL1 in these studies.

TABLE 5

| Genes | Forward Primer | Reverse Primer | Amplicon |
|---|---|---|---|
| PDZD2 | GCTGGGAATTCAG | TTGATTACCAGCAG | GCTGGGAATTCAGGGATGG |
| | GGATGG (SEQ ID | CTCATCTC (SEQ ID | CAGGCTGTCCTTAGGAGAT |
| | NO: 11) | NO: 12) | GAGCTGCTGGTAATCAA |
| | | | (SEQ ID NO: 13) |
| FAM107B | GCCAGGCAATATG | AACCTAAATGCCTC | GCCAGGCAATATGTCAGGA |
| | TCAGGA (SEQ ID | GAGCTG (SEQ ID NO: | CCTGATGTCATTTTCTGCTC |
| | NO: 14) | 15) | CAGCTCGAGGCATTTAGGT |
| | | | T (SEQ ID NO: 16) |
| HPCAL1 | GGCACCTGATCT | GGCCACTAGACCAT | GGCACCTTGATCTTGGACT |
| | TGGACTT (SEQ ID | GCC (SEQ ID NO: 18) | TCCAGCCCCCAGAACTGTA |
| | NO: 17) | | AGAGGGGCATGGTCTAGTG |
| | | | GCC (SEQ ID NO: 19) |

The investigation of novel splice variants in ccRCC also identified recurrent somatic splice variants in RNASET2 (TCGA=54% of tumors, TCC 50%, and CPTAC 35%). This gene represents the only human member of the T2 extracellular ribonucleases family and is believed to be overexpressed under cellular conditions of stress, such as hypoxia and cancer. This pathway is involved in several cancer types such as ovarian and melanoma where the RNASET2 protein has a non-cell autonomous oncosuppressive role. We examined the presence of two specific RNASET2 splice variants in several ccRCC patient cohorts and found their presence to be significantly associated with worse clinical outcomes, FIG. 8C. This significance remained after we controlled for important clinical variables such as tumor stage and patients age. These two possible neojunctions identified in these RNASET2 splice variants occur at the end of the coding sequence for the 24 amino acids signaling peptide, predicting to result in loss of this N-terminus region. Loss of such a signaling peptide would inhibit endoplasmic reticulum processing of the protein and prevent its secretion.

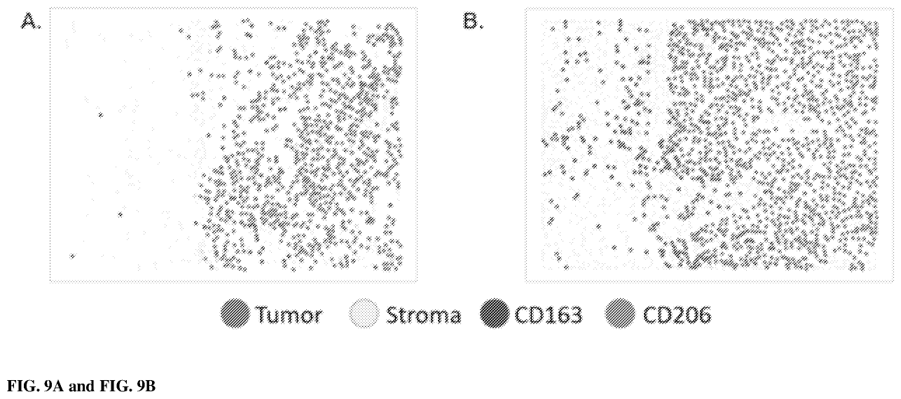

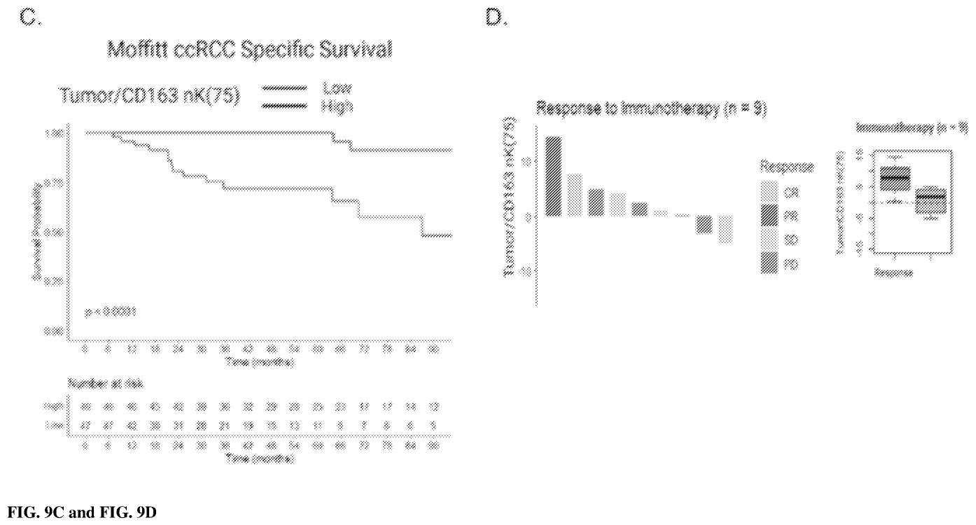

Several studies have demonstrated that extracellular RNASET2 can influence the tumor immune microenvironment (TIME) to facilitate polarization of macrophages away from the M2-like state (pro-tumor) to the M1-like state (anti-tumor). Interestingly, RNASET2 influence in the TIME is independent of the protein's catalytic activity. The pivotal role of macrophages in ccRCC has recently been highlighted by several studies and they support an interaction of M2-like macrophages with T-cells, causing them to become terminally exhausted. This interaction of M2-like macrophages and T-cells was associated with more advanced disease and worse survival in these studies. The study of the TIME in ccRCC (n=97) demonstrated that M-2 like macrophage clustering (CD163+) with stomal cell in the TIME was associated with inferior patient survival (FIG. 9 A,B,C). Within a in a small set of patients who received immunotherapy we saw a signal for poor response in those with increase CD163+/stromal clustering (FIG. 9D). This data supports an investigation of the presence of splice variants in RNASET2 and their association with a pro-tumor TIME in ccRCC.

Additionally, we saw moderately high levels of expression of the splice variant PDZD2 in breast cancer (200/336=60%) and endometrial cancer (170/311=55%). Wild type expression and over expression have been reportedly implicated in several cancers. Some studies of PDZD2 report its involvement in cell adhesion and is involved in regulating p53 activation. A germline single nucleotide polymorphism (SNP) with in PDZD2 has been associated with RCC among Chinese and European patients. We are not aware of any studies specific to the splicing of PDZD2 and its implication in cancer, or specifically ccRCC. Identification of splice variants was accomplished using the primers from Table 6.

TABLE 6

| Gene Name | Primer Name | Primer Sequences and Fluorecent Dyes |
|---|---|---|
| EGFR Wildtype | Ex19-RH-wildtype-F- | /5IABKFQ/CCG AAA GCC AAC AAG |
| | TAM | GAA ATC rCTC GAA/36-TAMSp/ |
| | | (SEQ ID NO: 20) |
| | Ex20-wildtype-R | GCC ATC ACG TAG GCT TCA T (SEQ |
| | | ID NO: 21) |
| EGFR Splice | In20-RH-splce-F-FAM | /5IABKFQ/CTT GAG GAA CTG GAT |
| Variant | | GGA GAA ArAG TAA T/36-FAM/ (SEQ |
| | | ID NO: 22) |
| | Ex21-splce-R | TCC AAG TAG TTC ATG CCC ATT |
| | | (SEQ ID NO: 23) |
| FAM107B Splice | FAM107B-splce-F-ROX | /5IAbRQ/GCC AGG CAA TAT GTC |
| Variant | | AGG ArCC TGA A/3Rox_N/ (SEQ ID |
| | | NO: 24) |
| | FAM107B-splce-R | AAC CTA AAT GCC TCG AGC TGrG |
| | | AGC AT/3SpC3/ (SEQ ID NO: 25) |
| PDZD2 Splice | PDZD2-splce-F-JOE | /5IABKFQ/GCT GGG AAT TCA GGG |
| Variant | | ATG GrCA GGC A/3Joe_N/ (SEQ ID |
| | | NO: 26) |
| | PDZD2-splce-R | TTG ATT ACC AGC AGC TCA TCT |
| | | CrCT AAG A/3SpC3/ (SEQ ID NO: 27) |

One important source of cell-free nucleic acids is extracellular RNAs (exRNAs), which are either freestanding or embedded inside circulating microvesicles (such as exosomes). exRNAs have been evaluated as biomarkers for monitoring dynamic changes of disease status and have also been studied for cancer detection, recurrence evaluation, and survival prediction. Exosomes are derived from multivesicular endosomes (MVEs) inside cytoplasm. Following fusion of the MVEs with cell membrane, exosomes are released into the extracellular area. The secreted exosomes are small enough to enter the blood circulation through the gaps between endothelial cells and disseminate to distant organs, where they are taken up by recipient cells. The internalized exosomes discharge their cargos, including proteins, miRNAs, lncRNAs, circRNAs, mRNAs and DNAs, which are functional in the new environment, resulting in changed physiological or pathological conditions of the recipient cells. These exRNAs are one main molecular component of exosomes and are known to cover a full spectrum of RNA species.

Recently, there has been early development of cell-free methylated DNA immunoprecipitation and high-throughput sequencing as a method of early ccRCC detection using patients' plasma. Though this method holds promise, it is still in development, requires prospective evaluation, and currently requires a large amount of expertise in cfDNA methylation profiling analysis. Studies show cancer cells shed cell-free RNA (cfRNA) to the blood. Primarily these studies have focused on microRNAs (miRNAs) as they tend to be stable and relatively prevalent in the plasma of patients. Yet the reproducibility and specificity of these studies across cohorts has been poor, possibly due to batch effect, methods in quantification and preanalytical processing conditions. More recently several groups have also published on the detection of known RNA transcripts among the cfRNA of healthy and cancer patients. These studies are seeking to identify cfRNA profiles among different cancer types but have not been evaluated in a clinical setting.

Compared to comprehensive transcriptomic characterization, identification of a specific short segment of RNA allows for a simplified quantification pipeline with minimal bioinformatic analysis. These short RNA segments in circulation from aberrant splice variants allow for one to resolve a single unique RNA back to tumor origin. One such technology that allows for very sensitive and specific detection of nucleic acid sequences is digital polymerase chain reaction (dPCR). This technology is a practical and scalable testing platform already employed for clinical use to detect alterations in EGFR and BRAF. Based on the RNA sequences of the three candidate splice variants we designed a set of primers and probes that specifically amplifies and quantifies these novel splice variants to develop a multiplexed dPCR assay. We collected patient plasma prior to planned surgical resection among patients with large renal masses and who had ccRCC. Matched tumor samples from each patient were also collected at the time of surgery. Plasma and tumor samples then underwent RNA extraction were analyzed using a multiplexed dPCR panel of the splice variants. We were able to detect splice variants among patient's plasma that correlated with those identified in matched tumor samples. The data for FAM107B detection among patient plasma, tumor, cell lines and controls are presented in FIG. 10. We found lower frequency of detection of PDZD2 and EGFR in plasma consistent with data in RNA seq analysis. As expected, the detection levels found in the plasma were lower than those found in matched tumors and in cell line controls. These results demonstrate the feasibility of investigating the three splice variants in ccRCC as a clinical biomarker.

a) Establishing Analytical Performance of the dPCR Assay by Spike-in Method

We can evaluate the analytical performance of the pre-designed multiplex dPCR assay using the synthesized RNA molecules. The RNA (which covers the entire amplicon) can be spiked into an RNA mixture that does not contain the respective aberrant splice variant. To optimize the dPCR condition, we can perform a series of dPCRs by varying concentration of primers/probes and annealing temperature. After selecting an optimal dPCR condition, we can then test the sensitivity of the assay for each variant by a series of dilutions of spike-in RNA. The limit of blank (LoB) and limit of detection (LoD) can be determined as the splice variant allele frequency using averaged replicate data. The LoB is defined as the splice variant allele frequency under which 95% of blank samples tested show no positivity. The LoD is defined as the splice variant allele frequency above which 95% of samples tested are distinguishable from a blank sample.

b) Multiplex dPCR Assay Characterization in Tumor Samples

Next to increase the robustness of our data we can evaluate detection of the candidate splice variants in ccRCC patients' tumors (n=30) using the dPCR assay and traditional bulk RNA sequencing. This analysis can also include non-ccRCC tumor controls (n=10). cDNA can be made by using the iScript Reverse Transcription Supermix (Bio-Rad Laboratories). dPCR can be performed in the reaction mixtures containing cDNA, primers/probes mix, and dPCR Probe Mastermix for Probes (Qiagen). The cycling protocol can follow the optimized condition from previous step. The absolute quantity of target RNA per sample (copies/μL) can be processed using QIAcuity dPCR software. These experiments can be used to determine the performance metrics of each assay with respect to specificity, sensitivity, and limits of detection in biological samples with respect to matched RNA seq data.

c) Multiplex dPCR Assay Characterization in Plasma Samples

Using the optimal dPCR condition determined above, we can further evaluate analytical performance of the multiplex dPCR assay (sensitivity, detection limits etc.) using plasma samples (including non-ccRCC controls). Because all patient's plasma have RNA-seq data from their tumor tissues, the status of the candidate splice variants for each patient is already known. We can calculate the sensitivity of the dPCR assay using the plasma samples using the following formula: sensitivity=number of true positives+(number of true positives+ number of false negatives). A sample size of 30 plasma samples from ccRCC patients will produce a one-sided 95% lower-limit confidence interval of dPCR sensitivity with the lower bound of 0.68, 0.72, and 0.75 when the sample sensitivity is 0.9 and the prevalence of a splice variant is 0.5 (low), 0.7 (middle), or 0.9 (high), respectively.

### 5. Example 5: Molecular and Clinical Implications of Recurrent Aberrant Splice Variants in Clear Cell Renal Cell Carcinoma

Given the lack of actionable genomic mutations in clear cell renal cell carcinoma (ccRCC), aberrant splice variants (SpVs) may be the avenue to new pathogenic mechanisms and biomarkers. We implemented a novel pipeline to screen for and select 16 SpVs frequent in and relatively specific to ccRCC. These include variants of suspected oncogenes and tumor suppressors. Bulk RNA-seq data of ccRCC primary tumors obtained from our institutional Total Cancer Care cohort (TCC), The Cancer Genome Atlas (TCGA) and the Clinical Proteomic Tumor Analysis Consortium (CPTAC) were analyzed for SpV expression in these samples. Genomic and molecular analyses identified interesting biological associations. We derived a SpV-based risk score trained on overall survival from the TCGA cohort and validated on the TCC and CPTAC cohorts. This study provides a template for identifying and characterizing disease-specific aberrant SpVs to aid discovery of new mechanisms and biomarkers.

To screen and select splice variants we used the selection scheme shown in FIG. 11, which resulted in the identification of 16 splice variants—epidermal growth factor receptor (EGFR) splice variant, Family With Sequence Similarity 107 Member B (FAM107B) splice variant, PDZ Domain Containing 2 (PDZD2) splice variant, Cordon-Bleu WH2 Repeat Protein Like 1 (COBLL1) splice variant, faciogenital dysplasia 1 (FGD1) splice variant, Galactose-3-O-Sulfotransferase 1 (GAL3ST1) splice variant, Hippocalcin Like 1 (HPCAL1) splice variant, KDEL (Lys-Asp-Glu-Leu) Containing 1 (KDELC1) splice variant, Methylcrotonyl-CoA Carboxylase Subunit 1 (MCCC1) splice variant, mevalonate kinase (MVK) splice variant, MVKb splice variant, Poly(A)-Specific Ribonuclease (PARN) splice variant, Protein Tyrosine Phosphatase Non-Receptor Type 14 (PTPN14) splice variant, Ribonuclease T2 beta (RNASET2β) splice variant, Solute carrier family 15, member 4 (SLC15A4) splice variant, and Synaptopodin (SYNPO) splice variant.

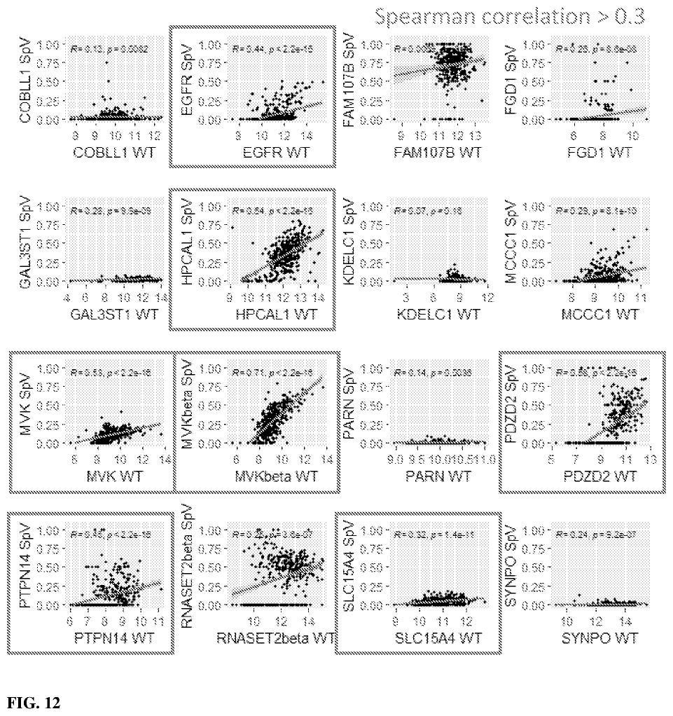

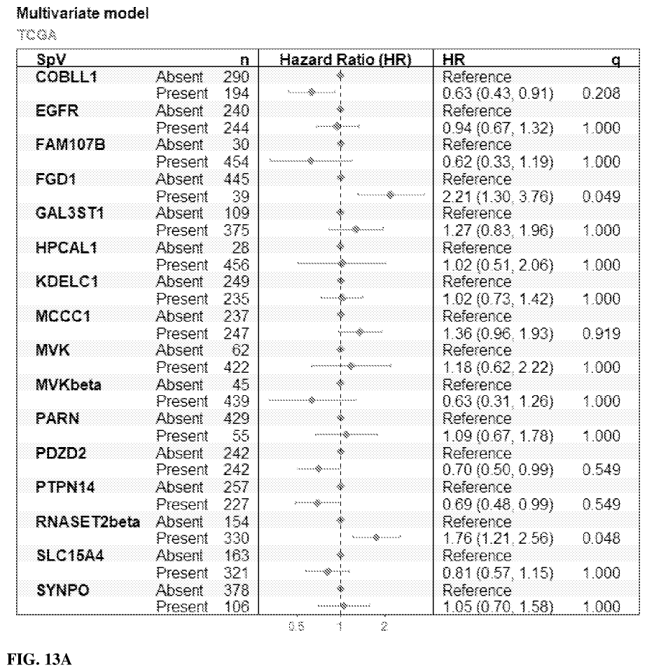

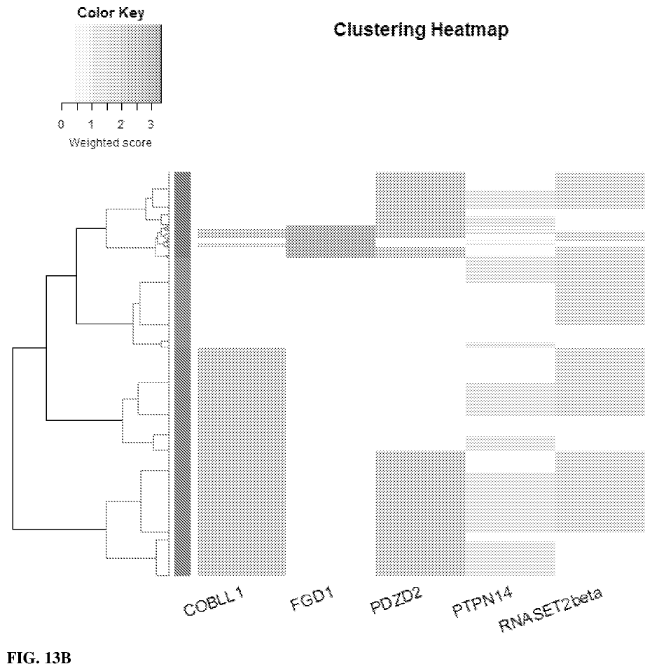

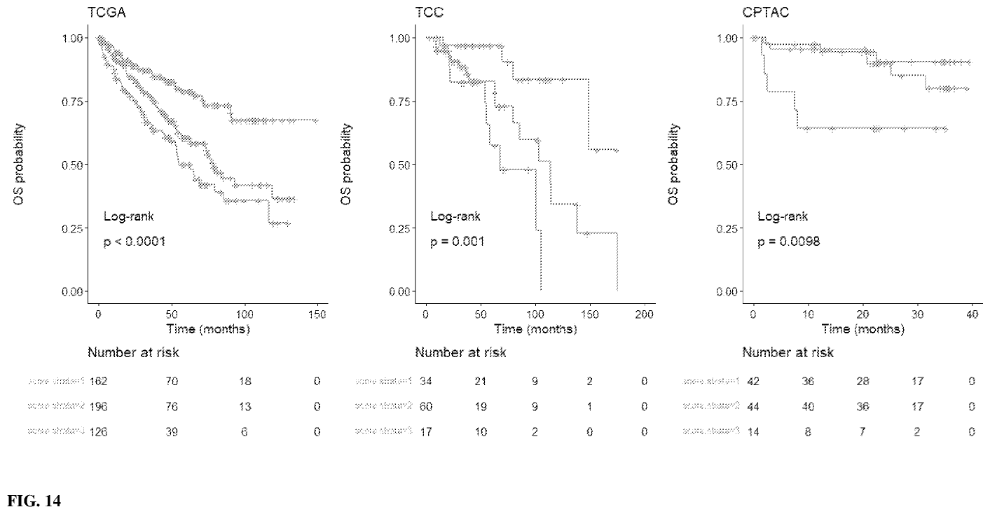

Next we examined the correlation between splice variant expression and wild-type expression in cancerous and non-cancerous tissue (FIG. 12). Using a hazard ratio and clustering heatmap (FIGS. 13A and 13B) we were able to establish risk associated with each splice variant. Next we looked and cancer specific survival (FIG. 14) associated with expression versus non-expression of certain splice variants (SpV). Using LASSO Cox regression analysis we learned that PDZD2-SpV (better OS; −2.5), COBLL1-SpV (better OS; −2.2), PTPN14-SpV (better OS; −1.3), RNASET2-SpV (worse OS; +1.9), FGD1-SpV (worse OS; +3.3). The risk score remained significant across all cohorts in multivariate Cox models (OS, CSS) adjusted for pathologic stage, age and gender.

Our novel pipeline selected 16 unique SpVs frequent in and relatively specific for ccRCC. Some are associated with proteins expressed in oncogenic pathways, suggesting a potential role in disease pathogenesis. Additionally, our SpV-based risk score is strongly associated with OS and CSS across multiple cohorts. This study provides a template for identifying and characterizing disease-specific aberrant SpVs to aid discovery of new mechanisms and biomarkers.

### E. REFERENCES

Acquati F, et al. Microenvironmental control of malignancy exerted by RNASET2, a widely conserved extracellular RNase. Proceedings of the National Academy of Sciences. 2011; 108(3):1104-9. doi: 10.1073/pnas.1013746108.
Acquati F, et al. Loss of function of Ribonuclease T2, an ancient and phylogenetically conserved RNase, plays a crucial role in ovarian tumorigenesis. Proceedings of the National Academy of Sciences. 2013; 110(20):8140-5. doi: 10.1073/pnas.1222079110.
Aran D, Hu Z, Butte A J. xCell: digitally portraying the tissue cellular heterogeneity landscape. Genome Biol. 2017; 18(1):220. Epub 2017/11/17. doi: 10.1186/s13059-017-1349-1. PubMed PMID: 29141660; PMCID: PMC5688663.
Armbruster D A, Pry T. Limit of blank, limit of detection and limit of quantitation. The Clinical biochemist Reviews. 2008; 29 Suppl 1(Suppl 1):549-52. Epub 2008/10/15. PubMed PMID: 18852857; PMCID: PMC2556583.
Baddeley A, Turner R. spatstat: An R package for analyzing spatial point patterns. J Stat Softw. 2005; 12(6):1-42. doi: DOI 10.18637/jss.v012.i06. PubMed PMID: WOS:000232806800001.
Barth D A, Drula R, Ott L, Fabris L, Slaby O, Calin G A, Pichler M. Circulating Non-coding RNAs in Renal Cell Carcinoma-Pathogenesis and Potential Implications as Clinical Biomarkers. Front Cell Dev Biol. 2020; 8:828-. doi: 10.3389/fcell.2020.00828. PubMed PMID: 33042985.
Berglund A, et al. Influence of gene expression on survival of clear cell renal cell carcinoma. Cancer Med. 2020; 9(22):8662-75. Epub 2020/09/29. doi: 10.1002/cam4.3475. PubMed PMID: 32986937; PMCID: PMC7666730.
Besag J. Contribution to the Discussion on Dr. Ripley's Paper. Journals of the Royal Statistical Society. 1977; B39:193-5.
Bi K, et al. Tumor and immune reprogramming during immunotherapy in advanced renal cell carcinoma. Cancer Cell. 2021.
Braun D A, et al. Interplay of somatic alterations and immune infiltration modulates response to PD-1 blockade in advanced clear cell renal cell carcinoma. Nature medicine. 2020; 26(6):909-18. Epub 2020/05/31. doi: 10.1038/s41591-020-0839-y. PubMed PMID: 32472114.
Braun D A, et al. Interplay of somatic alterations and immune infiltration modulates response to PD-1 blockade in advanced clear cell renal cell carcinoma. Nature medicine. 2020. doi: 10.1038/s41591-020-0839-y.
Braun D A, et al. Progressive immune dysfunction with advancing disease stage in renal cell carcinoma. Cancer Cell. 2021.
Carlo M I, et al. Genomic Alterations and Outcomes with VEGF-Targeted Therapy in Patients with Clear Cell Renal Cell Carcinoma. Kidney cancer. 2017; 1(1):49-56. Epub 2017/07/26. doi: 10.3233/kca-160003. PubMed PMID: 30334004; PMCID: PMC6179122.
Chakiryan N H, Jiang D D, Gillis K A, Green E, Hajiran A, Hugar L, Zemp L, Zhang J, Jain R K, Chahoud J, Spiess P E, Sexton W, Gilbert S M, Manley B J. Real-World Survival Outcomes Associated With First-Line Immunotherapy, Targeted Therapy, and Combination Therapy for Metastatic Clear Cell Renal Cell Carcinoma. JAMA Netw Open. 2021; 4(5):e2111329. Epub 2021/05/26.
Chakiryan N H, Kimmel G J, Kim Y, Hajiran A, Aydin A M, Zemp L, Katende E, Nguyen J, Lopez-Blanco N, Chahoud J, Spiess P E, Fournier M, Dhillon J, Wang L, Moran-Segura C, El-Kenawi A, Mule J, Altrock P M, Manley B J. Spatial clustering of CD68+ tumor associated macrophages with tumor cells is associated with worse overall survival in metastatic clear cell renal cell carcinoma. PloS one. 2021; 16(4):e0245415. Epub 2021/04/22.
Chakiryan N H, et al. Geospatial Cellular Distribution of Cancer-Associated Fibroblasts Significantly Impacts Clinical Outcomes in Metastatic Clear Cell Renal Cell Carcinoma. Cancers. 2021; 13(15):3743. PubMed PMID: doi:10.3390/cancersl3153743.
Chevinsky M, et al. Pathological Stage T3a Significantly Increases Disease Recurrence across All Tumor Sizes in Renal Cell Carcinoma. The Journal of urology. 2015; 194(2):310-5. Epub 2015/02/09. doi: 10.1016/j.juro.2015.02.013. PubMed PMID: 25676433.
Choueiri T K, Motzer R J. Systemic Therapy for Metastatic Renal-Cell Carcinoma. New England Journal of Medicine. 2017; 376(4):354-66. doi: 10.1056/NEJMra1601333. PubMed PMID: 28121507.
Danan-Gotthold M, Golan-Gerstl R, Eisenberg E, Meir K, Karni R, Levanon E Y. Identification of recurrent regulated alternative splicing events across human solid tumors. Nucleic Acids Res. 2015; 43(10):5130-44. Epub 2015/04/23. doi: 10.1093/nar/gkv210. PubMed PMID: 25908786.
Das S, Extracellular RNACC, Ansel K M, Bitzer M, Breakefield X O, Charest A, Galas D J, Gerstein M B, Gupta M, Milosavljevic A, McManus M T, Patel T, Raffai R L, Rozowsky J, Roth M E, Saugstad J A, Van Keuren-Jensen K, Weaver A M, Laurent L C. The Extracellular RNA Communication Consortium: Establishing Foundational Knowledge and Technologies for Extracellular RNA Research. Cell. 2019; 177(2):231-42. Epub 2019/04/06. doi: 10.1016/j.cell.2019.03.023. PubMed PMID: 30951667; PMCID: PMC6601620.
de Velasco G, Miao D, Voss M H, Hakimi A A, Hsieh J J, Tannir N M, Tamboli P, Appleman L J, Rathmell W K, Van Allen E M, Choueiri T K. Tumor Mutational Load and Immune Parameters across Metastatic Renal Cell Carcinoma Risk Groups. Cancer immunology research. 2016; 4(10):820-2. Epub 2016/08/20. doi: 10.1158/2326-6066.Cir-16-0110. PubMed PMID: 27538576; PMCID: PMC5050137.
Diaz de Leon A, Pedrosa I. Imaging and Screening of Kidney Cancer. Radiol Clin North Am. 2017; 55(6):1235-50. doi: 10.1016/j.rcl.2017.06.007. PubMed PMID: 28991563.
Dixon P M. Ripley's K function. In: Piegorsch AHE-SaWW, editor. Encyclopedia of Environmetrics2002. p. 1796-803.
Eggener S E, Rumble R B, Armstrong A J, Morgan T M, Crispino T, Cornford P, van der Kwast T, Grignon D J, Rai A J, Agarwal N, Klein E A, Den R B, Beltran H. Molecular Biomarkers in Localized Prostate Cancer: ASCO Guideline. J Clin Oncol. 2020; 38(13):1474-94. Epub 2019/12/13. doi: 10.1200/jco.19.02768. PubMed PMID: 31829902.
Engstrom P G, Steijger T, Sipos B, Grant G R, Kahles A, Ratsch G. Systematic evaluation of spliced alignment programs for RNA-seq data. Nat Methods. 2013; 10. doi: 10.1038/nmeth.2722.
Fabbri G, Holmes A B, Viganotti M, Scuoppo C, Belver L, Herranz D, Yan X-J, Kieso Y, Rossi D, Gaidano G, Chiorazzi N, Ferrando A A, Dalla-Favera R. Common nonmutational NOTCH1 activation in chronic lymphocytic leukemia. Proc Natl Acad Sci USA. 2017; 114(14):E2911-E9. Epub 2017/03/17. doi: 10.1073/pnas.1702564114. PubMed PMID: 28314854.
Farber N J, Kim C J, Modi P K, Hon J D, Sadimin E T, Singer E A. Renal cell carcinoma: the search for a reliable biomarker. Translational Cancer Research. 2017; 6(3):620-32.
Felsberg J, et al. Epidermal Growth Factor Receptor Variant III (EGFRvIII) Positivity in EGFR-Amplified Glioblastomas: Prognostic Role and Comparison between Primary and Recurrent Tumors. Clinical cancer research: an official journal of the American Association for Cancer Research. 2017; 23(22):6846-55. Epub 2017/09/01. doi: 10.1158/1078-0432.Ccr-17-0890. PubMed PMID: 28855349.
Ferrall-Fairbanks M C, Chakiryan N, Chobrutskiy B I, Kim Y, Teer J K, Berglund A, Muld J, Fournier M, Siegel E, Falasiri S, Arturo J F, Katende E N, Blanck G, Manley B J, Altrock P M. Quantifying T- and B-cell immune receptor distribution diversity to uncover their clinical relevance in clear cell renal cell carcinoma. medRxiv. 2021:2021.06.15.21258987. doi: 10.1101/2021.06.15.21258987.
Haase P. Spatial pattern analysis in ecology based on Ripley's K-function: Introduction and methods of edge correction. Journal of Vegetation Science. 1995; 6(4):575-82. doi: 10.2307/3236356.
Hajiran A, et al. Reconnaissance of tumor immune microenvironment spatial heterogeneity in metastatic renal cell carcinoma and correlation with immunotherapy response. Clin Exp Immunol. 2021; 204(1):96-106. Epub 2020/12/22. doi: 10.1111/cei.13567. PubMed PMID: 33346915; PMCID: PMC7944355.
Huang X, et al. Exosomal miR-1290 and miR-375 as prognostic markers in castration-resistant prostate cancer. European urology. 2015; 67(1):33-41. Epub 2014/08/19. doi: 10.1016/j.eururo.2014.07.035
Ji M, Zhao Z, Li Y, Xu P, Shi J, Li Z, Wang K, Huang X, Liu B. FBXO6-mediated RNASET2 ubiquitination and degradation governs the development of ovarian cancer. Cell Death & Disease. 2021; 12(4):317. doi: 10.1038/s41419-021-03580-4.
Joseph R W, Kapur P, Serie D J, Parasramka M, Ho T H, Cheville J C, Frenkel E, Parker A S, Brugarolas J. Clear Cell Renal Cell Carcinoma Subtypes Identified by BAP1 and PBRM1 Expression. The Journal of urology. 2016; 195(1):180-7. Epub 2015/08/25. doi: 10.1016/j.juro.2015.07.113. PubMed PMID: 26300218; PMCID: PMC5221690.
Kopreski M S, Benko F A, Kwak L W, Gocke C D. Detection of tumor messenger RNA in the serum of patients with malignant melanoma. Clinical cancer research: an official journal of the American Association for Cancer Research. 1999; 5(8):1961-5. Epub 1999/09/03. PubMed PMID: 10473072.
Krishna C, et al. Single-cell sequencing links multiregional immune landscapes and tissue-resident T cells in ccRCC to tumor topology and therapy efficacy. Cancer Cell. 2021; 39(5):662-77 e6. Epub 2021/04/17. doi: 10.1016/j.ccell.2021.03.007. PubMed PMID: 33861994.
Larson M H, Pan W, Kim H J, Mauntz R E, Stuart S M, Pimentel M, Zhou Y, Knudsgaard P, Demas V, Aravanis A M, Jamshidi A. A comprehensive characterization of the cell-free transcriptome reveals tissue- and subtype-specific biomarkers for cancer detection. Nat Commun. 2021; 12(1):2357. Epub 2021/04/23. doi: 10.1038/s41467-021-22444-1. PubMed PMID: 33883548; PMCID: PMC8060291 equity in the company. W. P., H. J. K., P. K. and A. M. A. are former employees of GRAIL, Inc. with equity in the company.
Lee C H, et al. Lenvatinib plus pembrolizumab in patients with either treatment-naive or previously treated metastatic renal cell carcinoma (Study 111/KEYNOTE-146): a phase 1b/2 study. The Lancet Oncology. 2021; 22(7):946-58. Epub 2021/06/19. doi: 10.1016/s1470-2045(21)00241-2. PubMed PMID: 34143969; PMCID: PMC8316679.
Lee I, Baxter D, Lee M Y, Scherler K, Wang K. The Importance of Standardization on Analyzing Circulating RNA. Molecular diagnosis & therapy. 2017; 21(3):259-68. Epub 2017/01/01. doi: 10.1007/s40291-016-0251-y. PubMed PMID: 28039578; PMCID: PMC5426982.
Lee S C, Abdel-Wahab O. Therapeutic targeting of splicing in cancer. Nature medicine. 2016; 22(9):976-86. Epub 2016/09/08. doi: 10.1038/nm.4165. PubMed PMID: 27603132; PMCID: PMC5644489.
Li C, He Q, Liang H, Cheng B, Li J, Xiong S, Zhao Y, Guo M, Liu Z, He J, Liang W. Diagnostic Accuracy of Droplet Digital PCR and Amplification Refractory Mutation System PCR for Detecting EGFR Mutation in Cell-Free DNA of Lung Cancer: A Meta-Analysis. Frontiers in oncology. 2020; 10:290-. doi: 10.3389/fonc.2020.00290. PubMed PMID: 32195189.
Manley B J, et al. Integration of Recurrent Somatic Mutations with Clinical Outcomes: A Pooled Analysis of 1049 Patients with Clear Cell Renal Cell Carcinoma. European urology focus. 2017; 3(4-5):421-7. Epub 2017/07/30. doi: 10.1016/j.euf.2016.06.015. PubMed PMID: 28753773; PMCID: PMC5650556.
Mapleson D, Venturini L, Kaithakottil G, Swarbreck D. Efficient and accurate detection of splice junctions from RNA-seq with Portcullis. Gigascience. 2018; 7(12):giyl31. doi: 10.1093/gigascience/giy131. PubMed PMID: 30418570.
Marcon E, Puech F. A typology of distance-based measures of spatial concentration. Regional Science and Urban Economics. 2017; 62:56-67.
Matera A G, Wang Z. A day in the life of the spliceosome. Nature reviews Molecular cell biology. 2014; 15(2):108-21. Epub 2014/01/24. doi: 10.1038/nrm3742. PubMed PMID: 24452469; PMCID: PMC4060434.
Max K E A, Bertram K, Akat K M, Bogardus K A, Li J, Morozov P, Ben-Dov I Z, Li X, Weiss Z R, Azizian A, Sopeyin A, Diacovo T G, Adamidi C, Williams Z, Tuschl T. Human plasma and serum extracellular small RNA reference profiles and their clinical utility. Proc Natl Acad Sci USA. 2018; 115(23):E5334-E43. Epub 2018/05/20. doi: 10.1073/pnas.1714397115. PubMed PMID: 29777089.
McDermott D F, et al. Clinical activity and molecular correlates of response to atezolizumab alone or in combination with bevacizumab versus sunitinib in renal cell carcinoma. Nature medicine. 2018; 24(6):749-57. Epub 2018/06/06. doi: 10.1038/s41591-018-0053-3. PubMed PMID: 29867230; PMCID: PMC6721896.
Miller B C, et al. Subsets of exhausted CD8+ T cells differentially mediate tumor control and respond to checkpoint blockade. Nature Immunology. 2019; 20(3):326-36. doi: 10.1038/s41590-019-0312-6.
Motzer R J, et al. NCCN Guidelines Insights: Kidney Cancer, Version 2.2020. Journal of the National Comprehensive Cancer Network: JNCCN. 2019; 17(11):1278-85. Epub 2019/11/07. doi: 10.6004/jnccn.2019.0054. PubMed PMID: 31693980.
Motzer R J, et al. Nivolumab plus ipilimumab versus sunitinib in first-line treatment for advanced renal cell carcinoma: extended follow-up of efficacy and safety results from a randomised, controlled, phase 3 trial. The Lancet Oncology. 2019; 20(10):1370-85.
Nghiem P T, et al. PD-1 Blockade with Pembrolizumab in Advanced Merkel-Cell Carcinoma. N Engl J Med. 2016; 374(26):2542-52. Epub 2016/04/19. doi: 10.1056/NEJMoa1603702. PubMed PMID: 27093365.
Nicholas C, et al. Geospatial characterization of immune cell distributions and dynamics across the microenvironment in renal cell carcinoma. Nature Portfolio. 2021. doi: 10.21203/rs.3.rs-847697/v1.
Nuzzo P V, et al. Detection of renal cell carcinoma using plasma and urine cell-free DNA methylomes. Nature medicine. 2020. doi: 10.1038/s41591-020-0933-1.
Pal S K, Sonpavde G, Agarwal N, Vogelzang N J, Srinivas S, Haas N B, Signoretti S, McGregor B A, Jones J, Lanman R B, Banks K C, Choueiri T K. Evolution of Circulating Tumor DNA Profile from First-line to Subsequent Therapy in Metastatic Renal Cell Carcinoma. European urology. 2017; 72(4):557-64. Epub 2017/04/18. doi: 10.1016/j.eururo.2017.03.046. PubMed PMID:28413127.
Perhavec A, Cerkovnik P, Novakovic S, Zgajnar J. The hTERT mRNA in plasma samples of early breast cancer patients, non-cancer patients and healthy individuals. Neoplasma. 2008; 55(6):549-54. Epub 2008/11/13. PubMed PMID: 18999886.
Reid A L, Freeman J B, Millward M, Ziman M, Gray E S. Detection of BRAF-V600E and V600K in melanoma circulating tumour cells by droplet digital PCR. Clinical biochemistry. 2015; 48(15):999-1002. Epub 2014/12/20. doi: 10.1016/j.clinbiochem.2014.12.007. PubMed PMID: 25523300.
Rini B I, et al. Pembrolizumab plus Axitinib versus Sunitinib for Advanced Renal-Cell Carcinoma. New England Journal of Medicine. 2019; 380(12):1116-27. doi: 10.1056/NEJMoa1816714. PubMed PMID: 30779529.
Ripley B D. Modelling Spatial Patterns. Journal of the Royal Statistical Society Series B (Methodological). 1977; 39(2):172-212.
Rodosthenous R S, Hutchins E, Reiman R, Yeri A S, Srinivasan S, Whitsett T G, Ghiran I, Silverman M G, Laurent L C, Van Keuren-Jensen K, Das S. Profiling Extracellular Long RNA Transcriptome in Human Plasma and Extracellular Vesicles for Biomarker Discovery. iScience. 2020; 23(6):101182. Epub 2020/06/09. doi: 10.1016/j.isci.2020.101182. PubMed PMID: 32512385; PMCID: PMC7283149.
S0302-2838(14)00687-3 [pii]. PubMed PMID: 25129854; PMCID: 4252606.
Seymour L, et al. iRECIST: guidelines for response criteria for use in trials testing immunotherapeutics. The Lancet Oncology. 2017; 18(3):e143-e52. Epub 2017/03/09. doi: 10.1016/s1470-2045(17)30074-8. PubMed PMID: 28271869; PMCID: PMC5648544.
Shah R V, Rong J, Larson M G, Yeri A, Ziegler 0, Tanriverdi K, Murthy V, Liu X, Xiao C, Pico A R, Huan T, Levy D, Lewis G D, Rosenzweig A, Vasan R S, Das S, Freedman J E. Associations of Circulating Extracellular RNAs With Myocardial Remodeling and Heart Failure. JAMA
Cardiol. 2018; 3(9):871-6. Epub 2018/08/10. doi: 10.1001/jamacardio.2018.2371. PubMed PMID: 30090932; PMCID: PMC6233646.
Skog J, Wurdinger T, van Rijn S, Meijer D H, Gainche L, Sena-Esteves M, Curry W T, Jr., Carter B S, Krichevsky A M, Breakefield X O. Glioblastoma microvesicles transport RNA and proteins that promote tumour growth and provide diagnostic biomarkers. Nat Cell Biol. 2008; 10(12):1470-6.
Soekmadji C, Hill A F, Wauben M H, Buzas E I, Di Vizio D, Gardiner C, Lotvall J, Sahoo S, Witwer K W. Towards mechanisms and standardization in extracellular vesicle and extracellular RNA studies: results of a worldwide survey. J Extracell Vesicles. 2018; 7(1):1535745. Epub 2018/10/30. doi: 10.1080/20013078.2018.1535745. PubMed PMID: 30370018; PMCID: PMC6201809.
Turajlic S, Swanton C, Boshoff C. Kidney cancer: The next decade. The Journal of Experimental Medicine. 2018. doi: 10.1084/jem.20181617.
van Niel G, Porto-Carreiro I, Simoes S, Raposo G. Exosomes: a common pathway for a specialized function. J Biochem. 2006; 140(1):13-21. Epub 2006/08/01. doi: 10.1093/jb/mvj128. PubMed PMID: 16877764.
Voss M H, Chen D, Marker M, Hakimi A A, Lee C H, Hsieh J J, Knox J J, Voi M, Motzer R J. Circulating biomarkers and outcome from a randomised phase II trial of sunitinib vs everolimus for patients with metastatic renal cell carcinoma. British journal of cancer. 2016; 114(6):642-9. Epub 2016/02/26.
Voss M H, Reising A, Cheng Y, Patel P, Marker M, Kuo F, Chan T A, Choueiri T K, Hsieh J J, Hakimi A A, Motzer R J. Genomically annotated risk model for advanced renal-cell carcinoma: a retrospective cohort study. The Lancet Oncology. 2018; 19(12):1688-98. Epub 2018/11/13. doi: 10.1016/s1470-2045(18)30648-x. PubMed PMID: 30416077; PMCID: PMC6701184.
Wang E T, Sandberg R, Luo S, Khrebtukova I, Zhang L, Mayr C, Kingsmore S F, Schroth G P, Burge C B. Alternative isoform regulation in human tissue transcriptomes. Nature. 2008; 456(7221):470-6. Epub 2008/11/04. doi: 10.1038/nature07509. PubMed PMID: 18978772; PMCID: PMC2593745.
Wang J, Zhang Y, Wei C, Gao X, Yuan P, Gan J, Li R, Liu Z, Wang T, Wang S, Liu J, Liu X. Prognostic Value of Androgen Receptor Splice Variant 7 in the Treatment of Metastatic Castration-Resistant Prostate Cancer: A Systematic Review and Meta-Analysis. Frontiers in oncology. 2020; 10:562504. Epub 2020/12/18. doi: 10.3389/fonc.2020.562504. PubMed PMID: 33330031; PMCID: PMC7735106.
Whiley P J, et al. Comparison of mRNA splicing assay protocols across multiple laboratories: recommendations for best practice in standardized clinical testing. Clinical chemistry. 2014; 60(2):341-52. Epub 2013/11/08. doi: 10.1373/clinchem.2013.210658. PubMed PMID: 24212087.
Yeri A, et al. Total Extracellular Small RNA Profiles from Plasma, Saliva, and Urine of Healthy Subjects. Sci Rep. 2017; 7:44061. Epub 2017/03/18. doi: 10.1038/srep44061. PubMed PMID: 28303895; PMCID: PMC5356006.
Yuan T, Huang X, Woodcock M, Du M, Dittmar R, Wang Y, Tsai S, Kohli M, Boardman L, Patel T, Wang L. Plasma extracellular RNA profiles in healthy and cancer patients. Sci Rep. 2016; 6:19413. Epub 2016/01/21. doi: 10.1038/srep19413 srep19413 [pii]. PubMed PMID: 26786760; PMCID: 4726401.
Zaman S, et al. Aberrant Epidermal Growth Factor Receptor RNA Splice Products Are Among the Most Frequent Somatic Alterations in Clear Cell Renal Cell Carcinoma and Are Associated with a Poor Response to Immunotherapy. European urology focus. 2019.
Zhou Z, Wu Q, Yan Z, Zheng H, Chen C J, Liu Y, Qi Z, Calandrelli R, Chen Z, Chien S, Su H I, Zhong S. Extracellular RNA in a single droplet of human serum reflects physiologic and disease states. Proc Natl Acad Sci USA. 2019; 116(38):19200-8. Epub 2019/09/05. doi: 10.1073/pnas.1908252116. PubMed PMID: 31481608; PMCID: PMC6754586.

1. A method of detecting resistance of a cancer to immune checkpoint blockade inhibition comprising obtaining a tissue sample from a subject with a cancer, performing droplet digital polymerase chain reaction (ddPCR) on the tissue sample comprising amplifying nucleic acid using primers specific for an epidermal growth factor receptor (EGFR) splice variant, Family With Sequence Similarity 107 Member B (FAM107B) splice variant, PDZ Domain Containing 2 (PDZD2) splice variant, Cordon-Bleu WH2 Repeat Protein Like 1 (COBLL1) splice variant, faciogenital dysplasia 1 (FGD1) splice variant, Galactose-3-O-Sulfotransferase 1 (GAL3ST1) splice variant, Hippocalcin Like 1 (HPCAL1) splice variant, KDEL (Lys-Asp-Glu-Leu) Containing 1 (KDELC1) splice variant, Methylcrotonyl-CoA Carboxylase Subunit 1 (MCCC1) splice variant, mevalonate kinase (MVK) splice variant, MVKb splice variant, Poly(A)-Specific Ribonuclease (PARN) splice variant, Protein Tyrosine Phosphatase Non-Receptor Type 14 (PTPN14) splice variant, Ribonuclease T2 beta (RNASET2p) splice variant, Solute carrier family 15, member 4 (SLC15A4) splice variant, and/or Synaptopodin (SYNPO) splice variant; wherein the presence of an EGFR, FAM107B, PDZD2, COBLL1, FGD1, GAL3ST1, HPCAL1, KDELC1, MCCC1, MVK, MVKβ, PARN, PTPN14, RNASET2P, SLC15A4, and/or SYNPO splice variant in the tissue sample indicates that the cancer is resistant to immune checkpoint blockade inhibition.

2. The method of detecting resistance of a cancer to immune checkpoint blockade inhibition of claim 1, further comprising treating with an anti-cancer therapy that is not an immune checkpoint blockade inhibitor when an EGFR, FAM107B, PDZD2, COBLL1, FGD1, GAL3ST1, HPCAL1, KDELC1, MCCC1, MVK, MVKβ, PARN, PTPN14, RNASET2P, SLC15A4, and/or SYNPO splice variant is detected.

3. A method of treating, inhibiting, reducing, and/or preventing a cancer and/or metastasis in a subject with a cancer comprising obtaining a tissue sample from the subject, performing droplet digital polymerase chain reaction (ddPCR) on the tissue sample comprising amplifying nucleic acid using primers specific for an epidermal growth factor receptor (EGFR) splice variant, Family With Sequence Similarity 107 Member B (FAM107B) splice variant, PDZ Domain Containing 2 (PDZD2) splice variant, Cordon-Bleu WH2 Repeat Protein Like 1 (COBLL1) splice variant, faciogenital dysplasia 1 (FGD1) splice variant, Galactose-3-O-Sulfotransferase 1 (GAL3ST1) splice variant, Hippocalcin Like 1 (HPCAL1) splice variant, KDEL (Lys-Asp-Glu-Leu) Containing 1 (KDELC1) splice variant, Methylcrotonyl-CoA Carboxylase Subunit 1 (MCCC1) splice variant, mevalonate kinase (MVK) splice variant, MVKb splice variant, Poly(A)-Specific Ribonuclease (PARN) splice variant, Protein Tyrosine Phosphatase Non-Receptor Type 14 (PTPN14) splice variant, Ribonuclease T2 beta (RNASET2p) splice variant, Solute carrier family 15, member 4 (SLC15A4) splice variant, and/or Synaptopodin (SYNPO) splice variant; wherein a decrease in the expression of an EGFR, FAM107B, and/or PDZD2 splice variant in the tissue sample indicates that the cancer is resistant to immune checkpoint blockade inhibition; and administering an anti-cancer therapy when EGFR, FAM107B, PDZD2, COBLL1, FGD1, GAL3ST1, HPCAL1, KDELC1, MCCC1, MVK, MVKβ, PARN, PTPN14, RNASET2P, SLC15A4, and/or SYNPO splice variant is detected.

4. (canceled)

5. The methods of claim 1, wherein the EGFR splice variant comprises a truncation of the EGF binding domain.

6. The methods of claim 5, wherein the EGFR splice variant comprises an alpha splice isoform of EGFR, a beta splice isoform of EGFR, or a gamma splice isoform of EGFR.

7. The method of claim 1, wherein the immune checkpoint blockade inhibitor to which the cancer is resistant is a PD-L1 blockade inhibitor.

8. (canceled)

9. The method of claim 3, wherein the anti-cancer therapy does not comprise the administration of immune checkpoint blockade inhibitors.

10. The method of claim 3, wherein the anti-cancer therapy does not comprise the administration of an immune checkpoint blockade inhibitor that targets PD-L1.

11. The method of claim 3, wherein the anti-cancer therapy comprises the administration of one or more of nivolumab, ipilimumab, and pembrolizumab.

12. (canceled)

13. A method detecting the presence of a metastatic cancer in a subject comprising obtaining a tissue sample from the subject, performing droplet digital polymerase chain reaction (ddPCR) on the tissue sample comprising amplifying nucleic acid using primers specific for an epidermal growth factor receptor (EGFR) splice variant, Family With Sequence Similarity 107 Member B (FAM107B) splice variant, PDZ Domain Containing 2 (PDZD2) splice variant, Cordon-Bleu WH2 Repeat Protein Like 1 (COBLL1) splice variant, faciogenital dysplasia 1 (FGD1) splice variant, Galactose-3-O-Sulfotransferase 1 (GAL3ST1) splice variant, Hippocalcin Like 1 (HPCAL1) splice variant, KDEL (Lys-Asp-Glu-Leu) Containing 1 (KDELC1) splice variant, Methylcrotonyl-CoA Carboxylase Subunit 1 (MCCC1) splice variant, mevalonate kinase (MVK) splice variant, MVKb splice variant, Poly(A)-Specific Ribonuclease (PARN) splice variant, Protein Tyrosine Phosphatase Non-Receptor Type 14 (PTPN14) splice variant, Ribonuclease T2 beta (RNASET2p) splice variant, Solute carrier family 15, member 4 (SLC15A4) splice variant, and/or Synaptopodin (SYNPO) splice variant; wherein a decrease in the expression of an EGFR, FAM107B, PDZD2, COBLL1, FGD1, GAL3ST1, HPCAL1, KDELC1, MCCC1, MVK, MVKβ, PARN, PTPN14, RNASET2P, SLC15A4, and/or SYNPO splice variant in the tissue sample indicates that the cancer has metastasized.

14. The methods of claim 13, wherein the tissue sample comprises whole blood, serum, plasma, cerebrospinal fluid, or urine.

15. The methods of claim 13, wherein the EGFR splice variant comprises a truncation of the EGF binding domain.

16. The methods of claim 15, wherein the EGFR splice variant comprises an alpha splice isoform of EGFR, a beta splice isoform of EGFR, or a gamma splice isoform of EGFR.

17. The method of claim 13, further comprising administering an anti-cancer agent to the subject.

18. The method of claim 17, wherein the anti-cancer agent is not an immune checkpoint blockade inhibitor.

19. The method of claim 17, wherein the anti-cancer therapy does not comprise the administration of an immune checkpoint blockade inhibitor that targets PD-L1.

20. (canceled)

21. (canceled)

22. A method of detecting recurrence of a cancer following immunotherapy, chemotherapy, radiation, and or tissue resection, the method comprising obtaining a tissue sample from the subject, performing droplet digital polymerase chain reaction (ddPCR) on the tissue sample comprising amplifying nucleic acid using primers specific for an epidermal growth factor receptor (EGFR) splice variant, Family With Sequence Similarity 107 Member B (FAM107B) splice variant, PDZ Domain Containing 2 (PDZD2) splice variant, Cordon-Bleu WH2 Repeat Protein Like 1 (COBLL1) splice variant, faciogenital dysplasia 1 (FGD1) splice variant, Galactose-3-O-Sulfotransferase 1 (GAL3ST1) splice variant, Hippocalcin Like 1 (HPCAL1) splice variant, KDEL (Lys-Asp-Glu-Leu) Containing 1 (KDELC1) splice variant, Methylcrotonyl-CoA Carboxylase Subunit 1 (MCCC1) splice variant, mevalonate kinase (MVK) splice variant, MVKb splice variant, Poly(A)-Specific Ribonuclease (PARN) splice variant, Protein Tyrosine Phosphatase Non-Receptor Type 14 (PTPN14) splice variant, Ribonuclease T2 beta (RNASET2p) splice variant, Solute carrier family 15, member 4 (SLC15A4) splice variant, and/or Synaptopodin (SYNPO) splice variant; wherein a decrease in the expression of an EGFR, FAM107B, PDZD2, COBLL1, FGD1, GAL3ST1, HPCAL1, KDELC1, MCCC1, MVK, MVKβ, PARN, PTPN14, RNASET2P, SLC15A4, and/or SYNPO splice variant in the tissue sample indicates that the cancer has recurred.

23. The method of claim 22, further comprising adjusting treatment and/or recommencing treatment of the cancer.

24. (canceled)

25. The methods of claim 3, wherein the EGFR splice variant comprises a truncation of the EGF binding domain.

26. The method of claim 3, wherein the immune checkpoint blockade inhibitor to which the cancer is resistant is a PD-L1 blockade inhibitor.


SEQUENCE LISTING (27 sequences)

SEQ ID NO: 1 - DNA, 20 bp, Artificial Sequence
  Synthetic construct
tattggctcccagtacctgc

SEQ ID NO: 2 - DNA, 21 bp, Artificial Sequence
  Synthetic construct
caagtagttcatgccctttgc

SEQ ID NO: 3 - DNA, 22 bp, Artificial Sequence
  Synthetic construct
tggtgtgtgcagatcgcaaagg

SEQ ID NO: 4 - DNA, 23 bp, Artificial Sequence
  Synthetic construct
catatacgagcacctctggactt

SEQ ID NO: 5 - DNA, 21 bp, Artificial Sequence
  Synthetic construct
ccaagtagttcatgcccatta

SEQ ID NO: 6 - DNA, 21 bp, Artificial Sequence
  Synthetic construct
ggaactggatggagaaaagtt

SEQ ID NO: 7 - DNA, 20 bp, Artificial Sequence
  Synthetic construct
tattggctcccagtacctgc

SEQ ID NO: 8 - DNA, 21 bp, Artificial Sequence
  Synthetic construct
ggtcctccaagtagttcatgc

SEQ ID NO: 9 - DNA, 23 bp, Artificial Sequence
  Synthetic construct
catatacgagcacctctggactt

SEQ ID NO: 10 - DNA, 21 bp, Artificial Sequence
  Synthetic construct
ggtcctccaagtagttcatgc

SEQ ID NO: 11 - DNA, 19 bp, Artificial Sequence
  Synthetic construct
gctgggaattcagggatgg

SEQ ID NO: 12 - DNA, 22 bp, Artificial Sequence
  Synthetic construct
ttgattaccagcagctcatctc

SEQ ID NO: 13 - DNA, 55 bp, Artificial Sequence
  Synthetic construct
gctgggaattcagggatggcaggctgtccttaggagatgagctgctggtaatcaa

SEQ ID NO: 14 - DNA, 19 bp, Artificial Sequence
  Synthetic construct
gccaggcaatatgtcagga

SEQ ID NO: 15 - DNA, 20 bp, Artificial Sequence
  Synthetic construct
aacctaaatgcctcgagctg

SEQ ID NO: 16 - DNA, 59 bp, Artificial Sequence
  Synthetic construct
gccaggcaatatgtcaggacctgatgtcattttctgctccagctcgaggcatttaggtt

SEQ ID NO: 17 - DNA, 20 bp, Artificial Sequence
  Synthetic construct
ggcaccttgatcttggactt

SEQ ID NO: 18 - DNA, 17 bp, Artificial Sequence
  Synthetic construct
ggccactagaccatgcc

SEQ ID NO: 19 - DNA, 60 bp, Artificial Sequence
  Synthetic construct
ggcaccttgatcttggacttccagcccccagaactgtaagaggggcatggtctagtggcc

SEQ ID NO: 20 - DNA, 28 bp, Artificial Sequence
  Synthetic construct
ccgaaagccaacaaggaaatcrctcgaa

SEQ ID NO: 21 - DNA, 19 bp, Artificial Sequence
  Synthetic construct
gccatcacgtaggcttcat

SEQ ID NO: 22 - DNA, 28 bp, Artificial Sequence
  Synthetic construct
cttgaggaactggatggagaaaragtaa

SEQ ID NO: 23 - DNA, 21 bp, Artificial Sequence
  Synthetic construct
tccaagtagttcatgcccatt

SEQ ID NO: 24 - DNA, 26 bp, Artificial Sequence
  Synthetic construct
gccaggcaatatgtcaggarcctgaa

SEQ ID NO: 25 - DNA, 27 bp, Artificial Sequence
  Synthetic construct
aacctaaatgcctcgagctgrgagcat

SEQ ID NO: 26 - DNA, 26 bp, Artificial Sequence
  Synthetic construct
gctgggaattcagggatggrcaggca

SEQ ID NO: 27 - DNA, 29 bp, Artificial Sequence
  Synthetic construct
ttgattaccagcagctcatctcrctaaga